(19) United States
(12) Patent Application Publication
(10) Pub. No.: US 2021/0161905 A1
(43) Pub. Date: Jun. 3, 2021

(54) Compositions for Preventing or Treating Dry Eye

(71) Applicant: CHONG KUN DANG PHARMACEUTICAL CORP., Seoul (KR)

(72) Inventors: Young II Choi, Gyeonggi-do (KR); Nina Ha, Gyeonggi-do (KR); Taek Hwan Shin, Gyeonggi-do (KR)

(21) Appl. No.: 17/046,402

(22) PCT Filed: Apr. 9, 2019
(86) PCT No.: PCT/KR2019/004227

§ 371 Date: Oct. 9, 2020

(30) Foreign Application Priority Data
Apr. 10, 2018 (KR) 10 2018 0041378

(51) Int. Cl. A61K 31/5375; A61K 9/00; A61P 27/04

(52) U.S. Cl. CPC A61K 31/5375; A61P 27/04; A61K 9/0053; A61K 9/0048

(57) ABSTRACT

The present invention relates to a pharmaceutical composition for preventing or treating dry eye, containing a compound represented by a formula I, an optical isomer thereof or a pharmaceutically acceptable salt thereof as an effective component, as well as a treatment method using the compound, and use of the compound in the manufacture of a medicament for treating dry eye. The pharmaceutical composition according to the present invention shows an excellent effect of preventing or treating dry eye.

### TECHNICAL FIELD

The present invention relates to a pharmaceutical composition for preventing or treating dry eye, comprising a compound represented by a formula I, an optical isomer thereof or a pharmaceutically acceptable salt thereof as an effective component, as well as a treatment method using the compound, and use of the compound in the manufacture of a medicament for treating dry eye.

### BACKGROUND ART

Dry eye is a multifactorial disease accompanied by an eye discomfort caused by a lack of tear volume or an abnormality of tear components, a visual disturbance, a unstability of a tear film, and a damage to a surface of an eyeball, wherein dry eye is known to be accompanied by an increase in osmolarity of the tear film and an inflammation on the surface of the eyeball (an increase in inflammatory cytokines).

Dry eye is a very common eye disease, which is thought to be associated with a short-distance work such as a computer use, a hormone abnormality or environmental factors such as air pollution. In particular, dry eye is known to increase with age and occur to women with a high frequency according to a decrease in sex hormone after menopause.

So far, however, dry eye has not been clarified about its pathological mechanism and a diagnostic method in proportion to a severity of symptoms has not been developed yet. Also, up to now, most of the therapeutic agents for dry eye have been an artificial tear, which is limited only to a temporal alleviation of symptoms, and thus there is an urgent need for developing an effective therapeutic agent.

Against these backdrops, the present inventors have made an every effort to develop a therapeutic agent for dry eye, and thus identified that a compound according to the present invention may be valuably used in preventing or treating dry eye, thereby completing the present invention.

### PRIOR ART REFERENCES

### Patent Document

(Patent Document 1) Korea Patent Application Publication No. 10-2014-0128886

### DISCLOSURE OF INVENTION

### Technical Problem

The objective of the present invention is to provide a pharmaceutical composition for preventing or treating dry eye, comprising a compound represented by a following formula I, an optical isomer thereof or a pharmaceutically acceptable salt thereof as an effective component.

Another objective of the present invention is to provide a method for treating dry eye, wherein the method comprises administering a therapeutically effective amount of the said compound.

Another objective of the present invention is to provide use of the compound in the manufacture of a medicament for treating dry eye.

### Solution to Problem

This will be described in detail as follows. Meanwhile, each description and embodiment form disclosed in the present invention may be applied to other descriptions and embodiment forms thereof, respectively. In other words, all combinations of various elements disclosed in the present invention fall within the scope of the present invention. Also, it cannot be seen that the scope of the present invention is limited to the specific description described below.

The present invention provides a pharmaceutical composition for preventing or treating dry eye, comprising a compound represented by a following formula I, an optical isomer thereof or a pharmaceutically acceptable salt thereof as an effective component:

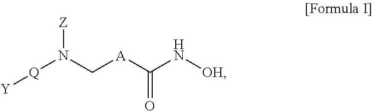

wherein in the Formula I,

A is

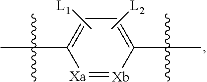

Xa and Xb are each independently CH or N,

L₁ and L₂ are each independently hydrogen, halogen, —CF₃ or —C₁₋₃ straight or branched chain alkyl,

Q is C(═O), S(═O)₂, S(═O) or C(═NH),

Y is selected from a following group:

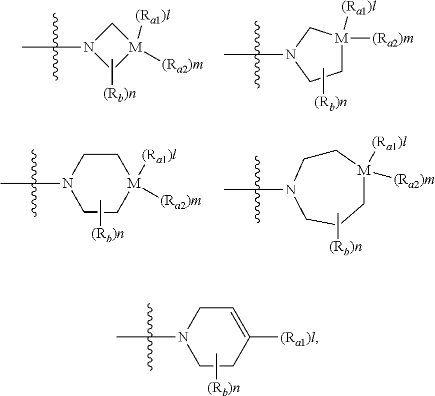

M is C, N, O, S or S(═O)₂, wherein, at this time, in case M is C, 1 and m are 1; in case M is N, 1 is 1 and m is 0; and in case M is O, S or S(═O)₂, 1 and m are 0,

Rₐ₁ and Rₐ₂ are each independently hydrogen; hydroxy; —C₁₋₄ straight or branched chain alkyl, which is unsubstituted or substituted with at least one halogen; —C₁₋₄ straight or branched chain alcohol; benzhydryl; —C₁₋₄ straight or branched chain alkyl, which is substituted with a saturated or unsaturated 5- to 7-membered heterocyclized compound comprising 1 to 3 heteroatoms of N, O or S as a ring member, wherein, at this time, the heterocyclized compound may be unsubstituted or at least one hydrogen may be optionally substituted with OH, OCH₃, CH₃, CH₂CH₃ or halogen; a saturated or unsaturated 5- to 7-membered heterocyclized compound comprising 1 to 3 heteroatoms of N, O or S as a ring member, wherein at this time, the heterocyclized compound may be unsubstituted or at least one hydrogen may be optionally substituted with OH, OCH₃, CH₃, CH₂CH₃ or halogen; phenyl, wherein it is unsubstituted or at least one hydrogen is substituted with halogen, C₁₋₄ alkoxy, C₁₋₂ alkyl or hydroxy; benzyl, wherein it is unsubstituted or at least one hydrogen is substituted with halogen, C₁₋₄ alkoxy, C₁₋₂ alkyl or hydroxy; —S(═O)₂CH₃; halogen; —C₁₋₆ straight or branched chain alkoxy; —C₂₋₆ alkoxyalkyl; —C(═O)Rₓ, wherein Rₓ is straight or branched chain C₁₋₃ alkyl or C₃₋₁₀ cycloalkyl;

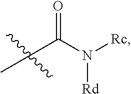

wherein R_{c} and R_{d} are each independently hydrogen, C₁₋₃ straight or branched chain alkyl; and

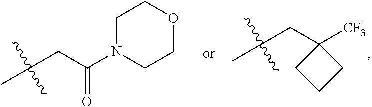 [SMILES: *CC(=O)N1CCOCC1 ; *CC1(C(F)(F)F)CCC1]

n is an integer of 0, 1 or 2,

R_{b} is hydrogen; hydroxy; —C₁₋₆ straight or branched chain alkyl, wherein it is unsubstituted or at least one hydrogen is substituted with halogen; —C(═O)CH₃; —C₁₋₄ straight or branched chain hydroxyalkyl; —C₁₋₆ straight or branched chain alkoxy; —C₂₋₆ straight or branched chain alkoxyalkyl; —CF₃; halogen; or

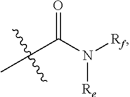

Rₑ and R_{f} are each independently hydrogen or —C₁₋₃ straight or branched chain alkyl,

Z is selected from a following group:

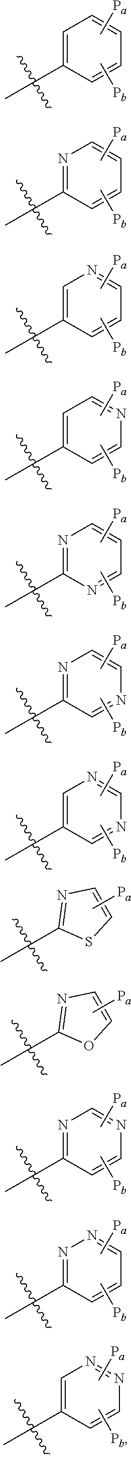

Pₐ and P_{b} are each independently

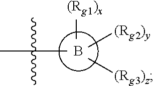

hydrogen; hydroxy; —C₁₋₄ straight or branched chain alkyl, wherein it is unsubstituted or at least one hydrogen is substituted with halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol,

wherein

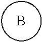

is phenyl, pyridine, pyrimidine, thiazole, indole, indazole, piperazine, quinoline, furan, tetrahydropyridine, piperidine or a ring selected from a following group:

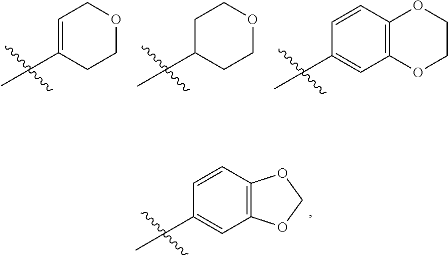 [SMILES: *C1=CCOCC1 ; *C1CCOCC1 ; *c1ccc2c(c1)OCCO2 ; *c1ccc2c(c1)OCO2]

x, y and z are each independently an integer of 0 or 1,

R_{g1}, R_{g2} and R_{g3} are each independently hydrogen; hydroxy; —C₁₋₃ alkyl; —CF₃; —C₁₋₆ straight or branched chain alkoxy; —C₂₋₆ straight or branched chain alkyl alkoxy; —C(═O)CH₃; —C₁₋₄ straight or branched chain hydroxyalkyl; —N(CH₃)₂; halogen; phenyl; —S((═O)₂)CH₃; or selected from a following group:

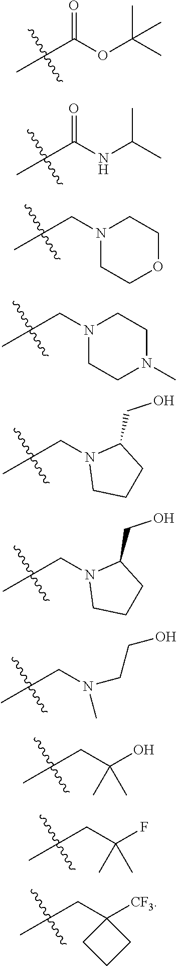

According to a specific embodiment of the present invention, the compound represented by the formula I above is a compound described in a following table 1 to table 12:

TABLE 1

| Compound | Structure |
|---|---|
| 252 | 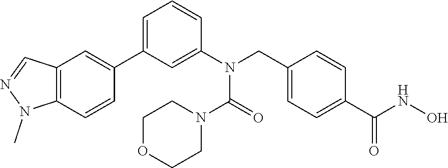 [SMILES: Cn1ncc2cc(-c3cccc(N(Cc4ccc(C(=O)NO)cc4)C(=O)N4CCOCC4)c3)ccc21] |
| 253 | 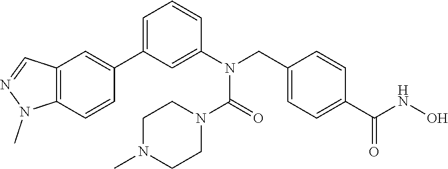 [SMILES: CN1CCN(C(=O)N(Cc2ccc(C(=O)NO)cc2)c2cccc(-c3ccc4c(cnn4C)c3)c2)CC1] |
| 254 | 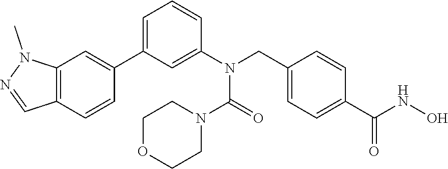 [SMILES: Cn1ncc2ccc(-c3cccc(N(Cc4ccc(C(=O)NO)cc4)C(=O)N4CCOCC4)c3)cc21] |
| 255 | 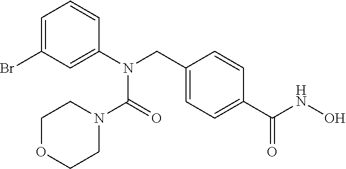 [SMILES: O=C(NO)c1ccc(CN(C(=O)N2CCOCC2)c2cccc(Br)c2)cc1] |
| 256 | 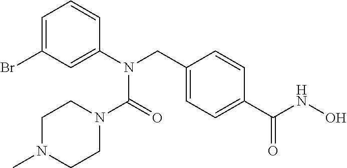 [SMILES: CN1CCN(C(=O)N(Cc2ccc(C(=O)NO)cc2)c2cccc(Br)c2)CC1] |
| 260 | 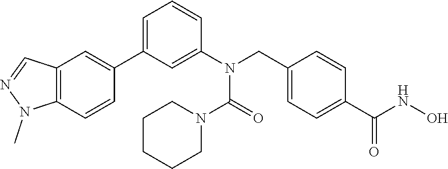 [SMILES: Cn1ncc2cc(-c3cccc(N(Cc4ccc(C(=O)NO)cc4)C(=O)N4CCCCC4)c3)ccc21] |
| 261 | 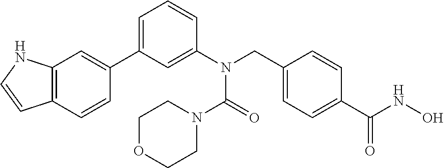 [SMILES: O=C(NO)c1ccc(CN(C(=O)N2CCOCC2)c2cccc(-c3ccc4cc[nH]c4c3)c2)cc1] |
| 262 | 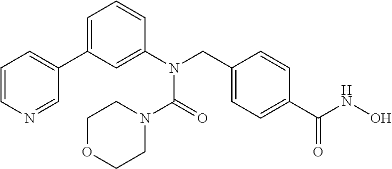 [SMILES: O=C(NO)c1ccc(CN(C(=O)N2CCOCC2)c2cccc(-c3cccnc3)c2)cc1] |
| 263 | 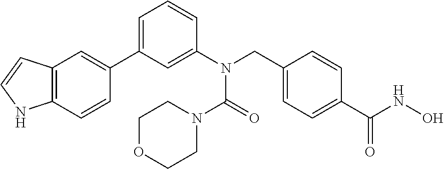 [SMILES: O=C(NO)c1ccc(CN(C(=O)N2CCOCC2)c2cccc(-c3ccc4[nH]ccc4c3)c2)cc1] |
| 279 | 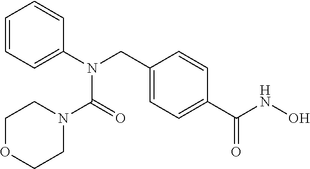 [SMILES: O=C(NO)c1ccc(CN(C(=O)N2CCOCC2)c2ccccc2)cc1] |
| 280 | 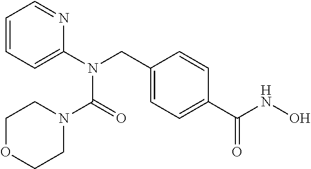 [SMILES: O=C(NO)c1ccc(CN(C(=O)N2CCOCC2)c2ccccn2)cc1] |
| 281 | 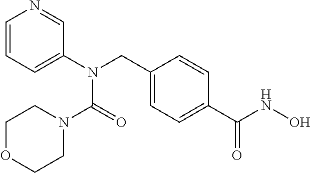 [SMILES: O=C(NO)c1ccc(CN(C(=O)N2CCOCC2)c2cccnc2)cc1] |
| 309 | 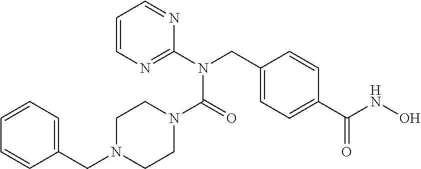 [SMILES: O=C(NO)c1ccc(CN(C(=O)N2CCN(Cc3ccccc3)CC2)c2ncccn2)cc1] |
| 311 | 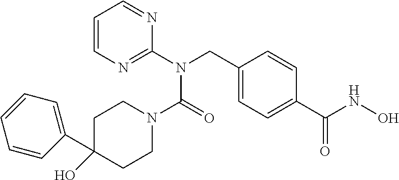 [SMILES: O=C(NO)c1ccc(CN(C(=O)N2CCC(O)(c3ccccc3)CC2)c2ncccn2)cc1] |

TABLE 2

| Compound | Structure |
|---|---|
| 312 | 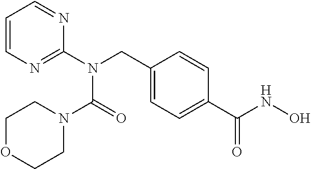 [SMILES: O=C(NO)c1ccc(CN(C(=O)N2CCOCC2)c2ncccn2)cc1] |
| 313 | 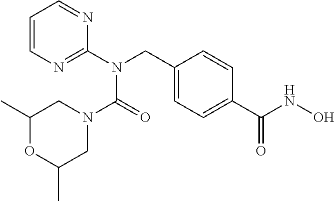 [SMILES: CC1CN(C(=O)N(Cc2ccc(C(=O)NO)cc2)c2ncccn2)CC(C)O1] |
| 329 | 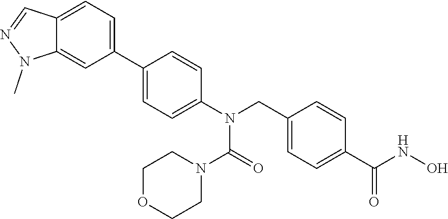 [SMILES: Cn1ncc2ccc(-c3ccc(N(Cc4ccc(C(=O)NO)cc4)C(=O)N4CCOCC4)cc3)cc21] |
| 330 | 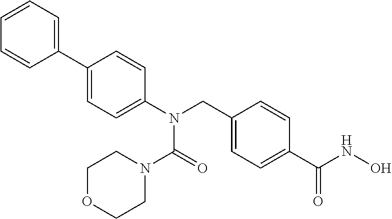 [SMILES: O=C(NO)c1ccc(CN(C(=O)N2CCOCC2)c2ccc(-c3ccccc3)cc2)cc1] |
| 331 | 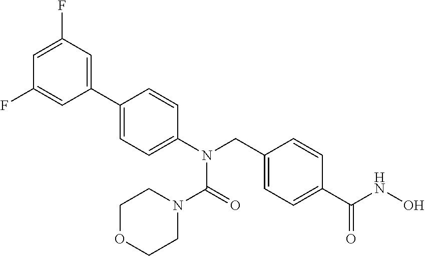 [SMILES: O=C(NO)c1ccc(CN(C(=O)N2CCOCC2)c2ccc(-c3cc(F)cc(F)c3)cc2)cc1] |
| 332 | 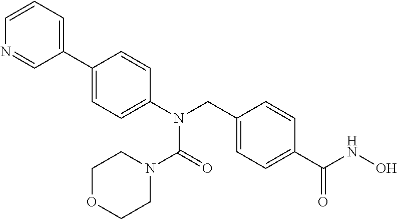 [SMILES: O=C(NO)c1ccc(CN(C(=O)N2CCOCC2)c2ccc(-c3cccnc3)cc2)cc1] |
| 333 | 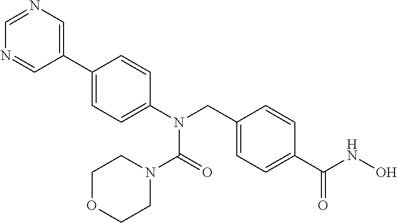 [SMILES: O=C(NO)c1ccc(CN(C(=O)N2CCOCC2)c2ccc(-c3cncnc3)cc2)cc1] |
| 334 | 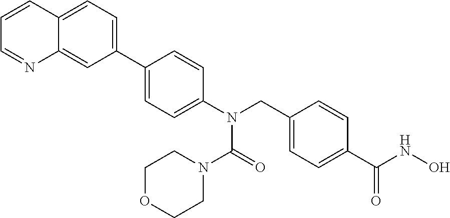 [SMILES: O=C(NO)c1ccc(CN(C(=O)N2CCOCC2)c2ccc(-c3ccc4cccnc4c3)cc2)cc1] |
| 335 | 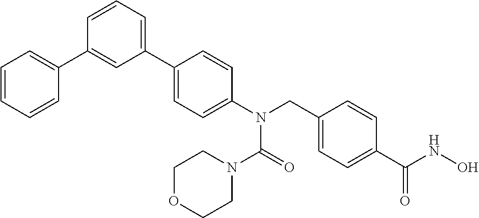 [SMILES: O=C(NO)c1ccc(CN(C(=O)N2CCOCC2)c2ccc(-c3cccc(-c4ccccc4)c3)cc2)cc1] |
| 336 | 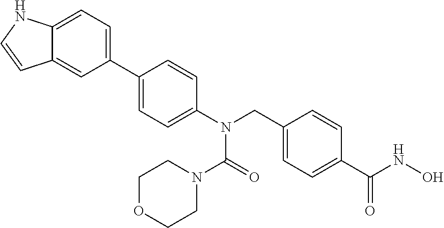 [SMILES: O=C(NO)c1ccc(CN(C(=O)N2CCOCC2)c2ccc(-c3ccc4[nH]ccc4c3)cc2)cc1] |
| 337 | 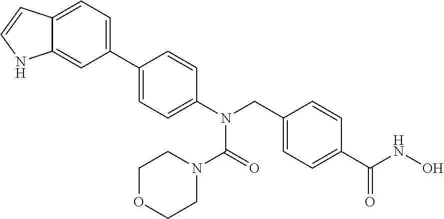 [SMILES: O=C(NO)c1ccc(CN(C(=O)N2CCOCC2)c2ccc(-c3ccc4cc[nH]c4c3)cc2)cc1] |
| 338 | 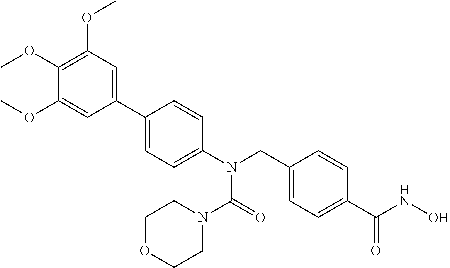 [SMILES: COc1cc(-c2ccc(N(Cc3ccc(C(=O)NO)cc3)C(=O)N3CCOCC3)cc2)cc(OC)c1OC] |
| 339 | 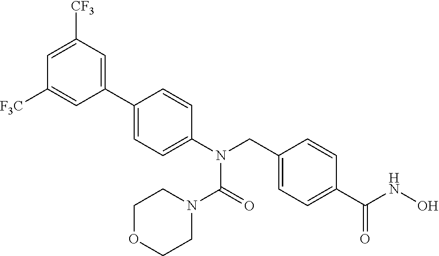 [SMILES: O=C(NO)c1ccc(CN(C(=O)N2CCOCC2)c2ccc(-c3cc(C(F)(F)F)cc(C(F)(F)F)c3)cc2)cc1] |
| 340 | 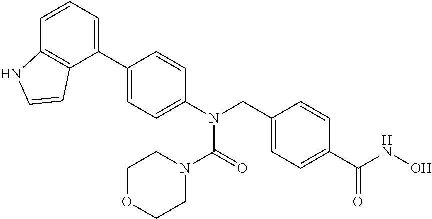 [SMILES: O=C(NO)c1ccc(CN(C(=O)N2CCOCC2)c2ccc(-c3cccc4[nH]ccc34)cc2)cc1] |

TABLE 3

| Compound | Structure |
|---|---|
| 341 | 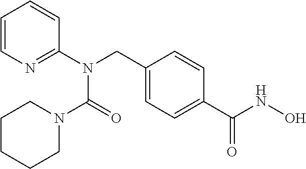 [SMILES: O=C(NO)c1ccc(CN(C(=O)N2CCCCC2)c2ccccn2)cc1] |
| 342 | 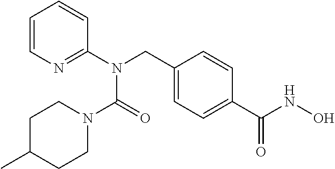 [SMILES: CC1CCN(C(=O)N(Cc2ccc(C(=O)NO)cc2)c2ccccn2)CC1] |
| 343 | 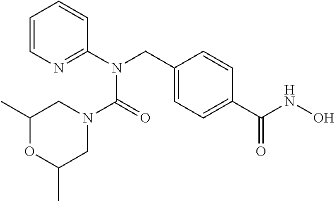 [SMILES: CC1CN(C(=O)N(Cc2ccc(C(=O)NO)cc2)c2ccccn2)CC(C)O1] |
| 352 | 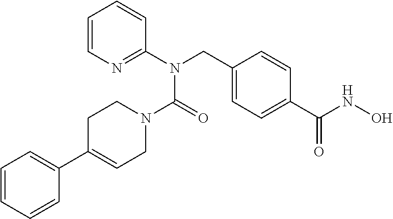 [SMILES: O=C(NO)c1ccc(CN(C(=O)N2CC=C(c3ccccc3)CC2)c2ccccn2)cc1] |
| 353 | 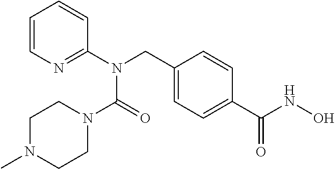 [SMILES: CN1CCN(C(=O)N(Cc2ccc(C(=O)NO)cc2)c2ccccn2)CC1] |
| 354 | 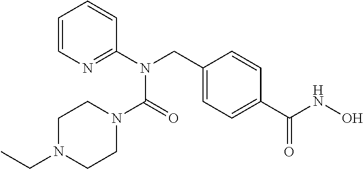 [SMILES: CCN1CCN(C(=O)N(Cc2ccc(C(=O)NO)cc2)c2ccccn2)CC1] |
| 355 | 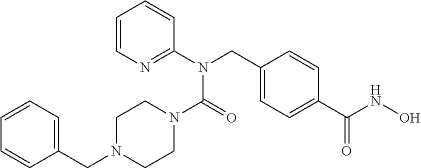 [SMILES: O=C(NO)c1ccc(CN(C(=O)N2CCN(Cc3ccccc3)CC2)c2ccccn2)cc1] |
| 356 | 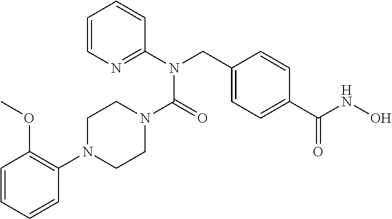 [SMILES: COc1ccccc1N1CCN(C(=O)N(Cc2ccc(C(=O)NO)cc2)c2ccccn2)CC1] |
| 357 | 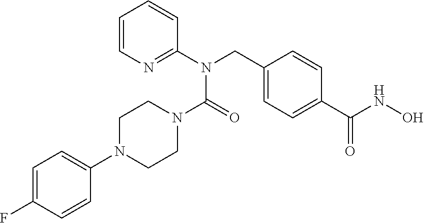 [SMILES: O=C(NO)c1ccc(CN(C(=O)N2CCN(c3ccc(F)cc3)CC2)c2ccccn2)cc1] |
| 358 | 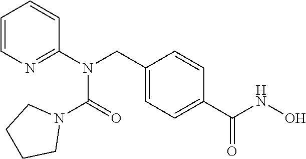 [SMILES: O=C(NO)c1ccc(CN(C(=O)N2CCCC2)c2ccccn2)cc1] |
| 370 | 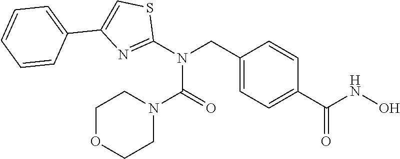 [SMILES: O=C(NO)c1ccc(CN(C(=O)N2CCOCC2)c2nc(-c3ccccc3)cs2)cc1] |
| 371 | 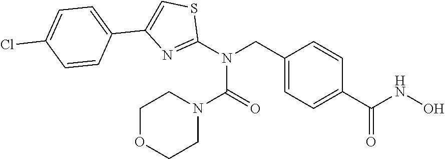 [SMILES: O=C(NO)c1ccc(CN(C(=O)N2CCOCC2)c2nc(-c3ccc(Cl)cc3)cs2)cc1] |
| 372 | 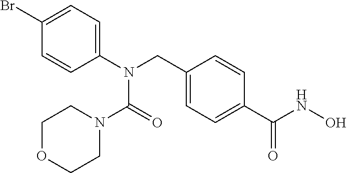 [SMILES: O=C(NO)c1ccc(CN(C(=O)N2CCOCC2)c2ccc(Br)cc2)cc1] |
| 374 | 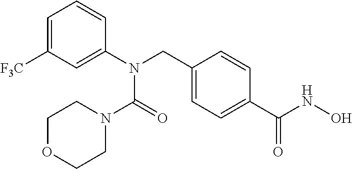 [SMILES: O=C(NO)c1ccc(CN(C(=O)N2CCOCC2)c2cccc(C(F)(F)F)c2)cc1] |

TABLE 4

| Compound | Structure |
|---|---|
| 376 | 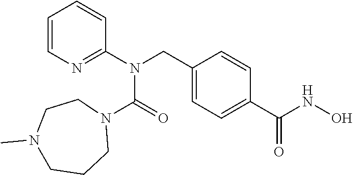 [SMILES: CN1CCCN(C(=O)N(Cc2ccc(C(=O)NO)cc2)c2ccccn2)CC1] |
| 377 | 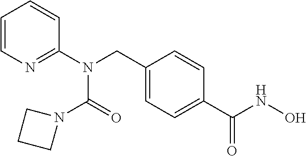 [SMILES: O=C(NO)c1ccc(CN(C(=O)N2CCC2)c2ccccn2)cc1] |
| 379 | 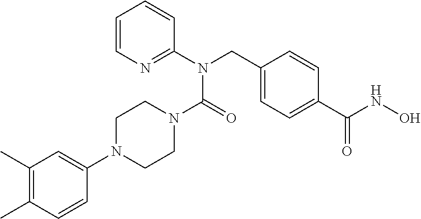 [SMILES: Cc1ccc(N2CCN(C(=O)N(Cc3ccc(C(=O)NO)cc3)c3ccccn3)CC2)cc1C] |
| 380 | 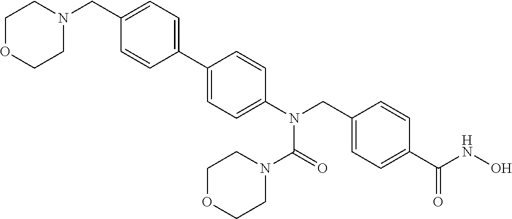 [SMILES: O=C(NO)c1ccc(CN(C(=O)N2CCOCC2)c2ccc(-c3ccc(CN4CCOCC4)cc3)cc2)cc1] |
| 381 | 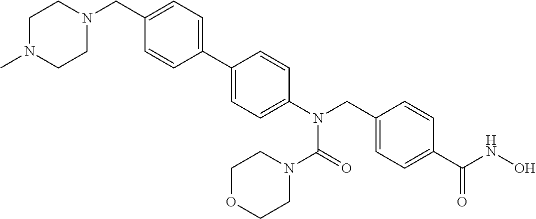 [SMILES: CN1CCN(Cc2ccc(-c3ccc(N(Cc4ccc(C(=O)NO)cc4)C(=O)N4CCOCC4)cc3)cc2)CC1] |
| 382 | 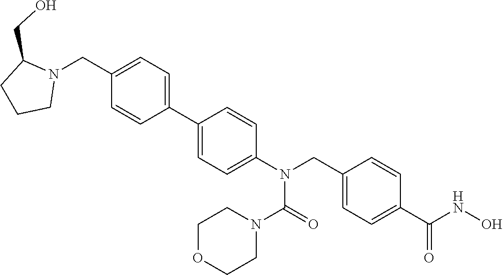 [SMILES: O=C(NO)c1ccc(CN(C(=O)N2CCOCC2)c2ccc(-c3ccc(CN4CCC[C@H]4CO)cc3)cc2)cc1] |
| 383 | 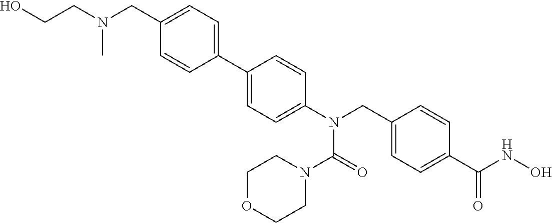 [SMILES: CN(CCO)Cc1ccc(-c2ccc(N(Cc3ccc(C(=O)NO)cc3)C(=O)N3CCOCC3)cc2)cc1] |
| 385 | 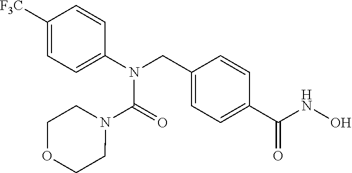 [SMILES: O=C(NO)c1ccc(CN(C(=O)N2CCOCC2)c2ccc(C(F)(F)F)cc2)cc1] |
| 386 | 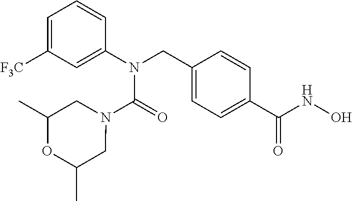 [SMILES: CC1CN(C(=O)N(Cc2ccc(C(=O)NO)cc2)c2cccc(C(F)(F)F)c2)CC(C)O1] |
| 389 | 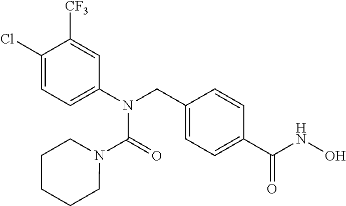 [SMILES: O=C(NO)c1ccc(CN(C(=O)N2CCCCC2)c2ccc(Cl)c(C(F)(F)F)c2)cc1] |
| 390 | 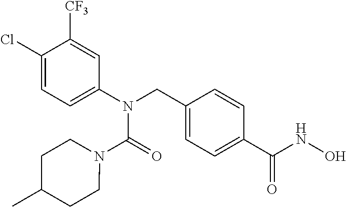 [SMILES: CC1CCN(C(=O)N(Cc2ccc(C(=O)NO)cc2)c2ccc(Cl)c(C(F)(F)F)c2)CC1] |
| 391 | 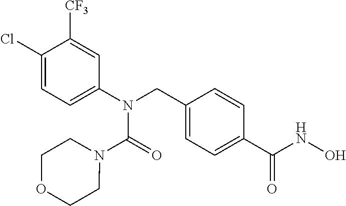 [SMILES: O=C(NO)c1ccc(CN(C(=O)N2CCOCC2)c2ccc(Cl)c(C(F)(F)F)c2)cc1] |
| 392 | 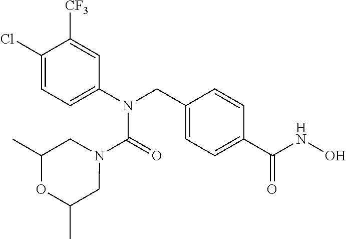 [SMILES: CC1CN(C(=O)N(Cc2ccc(C(=O)NO)cc2)c2ccc(Cl)c(C(F)(F)F)c2)CC(C)O1] |
| 393 | 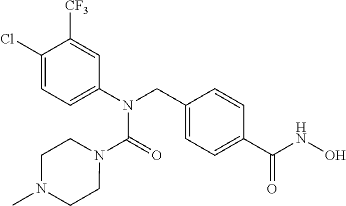 [SMILES: CN1CCN(C(=O)N(Cc2ccc(C(=O)NO)cc2)c2ccc(Cl)c(C(F)(F)F)c2)CC1] |

TABLE 5

| Compound | Structure |
|---|---|
| 394 | 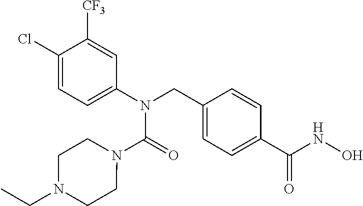 [SMILES: CCN1CCN(C(=O)N(Cc2ccc(C(=O)NO)cc2)c2ccc(Cl)c(C(F)(F)F)c2)CC1] |
| 395 | 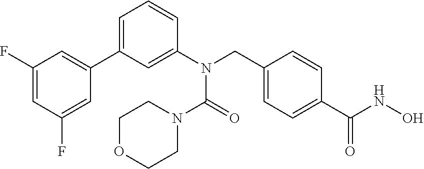 [SMILES: O=C(NO)c1ccc(CN(C(=O)N2CCOCC2)c2cccc(-c3cc(F)cc(F)c3)c2)cc1] |
| 396 | 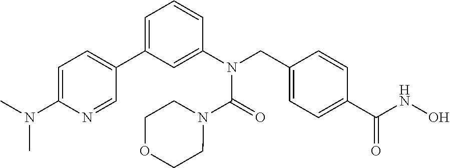 [SMILES: CN(C)c1ccc(-c2cccc(N(Cc3ccc(C(=O)NO)cc3)C(=O)N3CCOCC3)c2)cn1] |
| 397 | 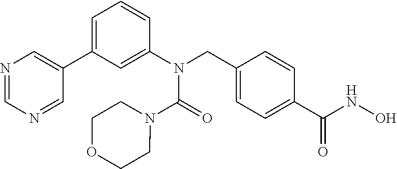 [SMILES: O=C(NO)c1ccc(CN(C(=O)N2CCOCC2)c2cccc(-c3cncnc3)c2)cc1] |
| 398 | 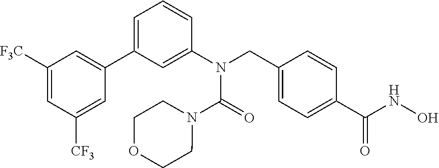 [SMILES: O=C(NO)c1ccc(CN(C(=O)N2CCOCC2)c2cccc(-c3cc(C(F)(F)F)cc(C(F)(F)F)c3)c2)cc1] |
| 399 | 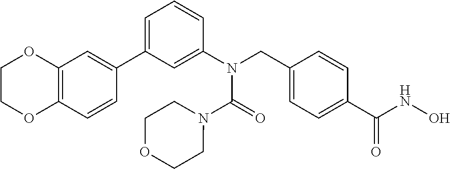 [SMILES: O=C(NO)c1ccc(CN(C(=O)N2CCOCC2)c2cccc(-c3ccc4c(c3)OCCO4)c2)cc1] |
| 400 | 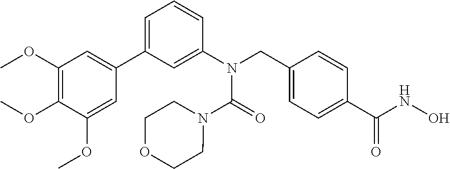 [SMILES: COc1cc(-c2cccc(N(Cc3ccc(C(=O)NO)cc3)C(=O)N3CCOCC3)c2)cc(OC)c1OC] |
| 401 | 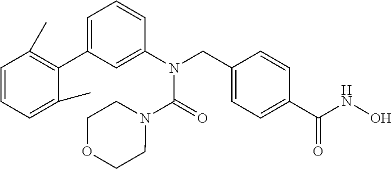 [SMILES: Cc1cccc(C)c1-c1cccc(N(Cc2ccc(C(=O)NO)cc2)C(=O)N2CCOCC2)c1] |
| 402 | 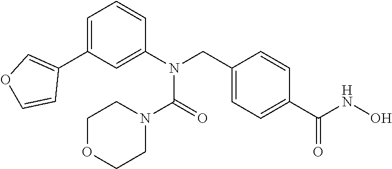 [SMILES: O=C(NO)c1ccc(CN(C(=O)N2CCOCC2)c2cccc(-c3ccoc3)c2)cc1] |
| 403 | 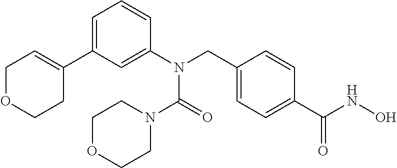 [SMILES: O=C(NO)c1ccc(CN(C(=O)N2CCOCC2)c2cccc(C3=CCOCC3)c2)cc1] |
| 404 | 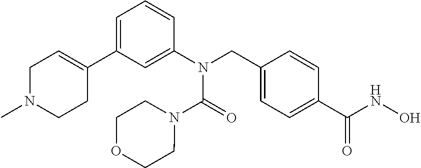 [SMILES: CN1CC=C(c2cccc(N(Cc3ccc(C(=O)NO)cc3)C(=O)N3CCOCC3)c2)CC1] |
| 405 | 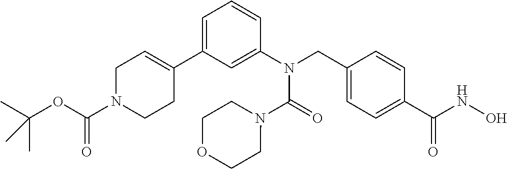 [SMILES: CC(C)(C)OC(=O)N1CC=C(c2cccc(N(Cc3ccc(C(=O)NO)cc3)C(=O)N3CCOCC3)c2)CC1] |
| 413 | 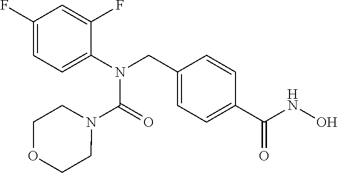 [SMILES: O=C(NO)c1ccc(CN(C(=O)N2CCOCC2)c2ccc(F)cc2F)cc1] |
| 414 | 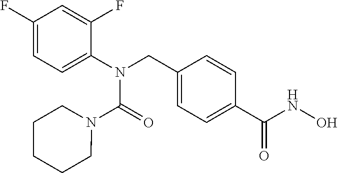 [SMILES: O=C(NO)c1ccc(CN(C(=O)N2CCCCC2)c2ccc(F)cc2F)cc1] |

TABLE 6

| Compound | Structure |
|---|---|
| 415 | 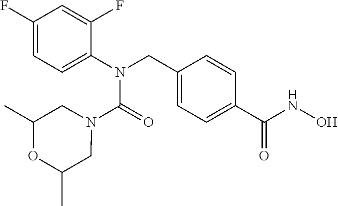 [SMILES: CC1CN(C(=O)N(Cc2ccc(C(=O)NO)cc2)c2ccc(F)cc2F)CC(C)O1] |
| 416 | 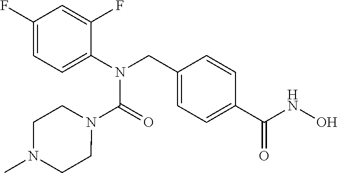 [SMILES: CN1CCN(C(=O)N(Cc2ccc(C(=O)NO)cc2)c2ccc(F)cc2F)CC1] |
| 418 | 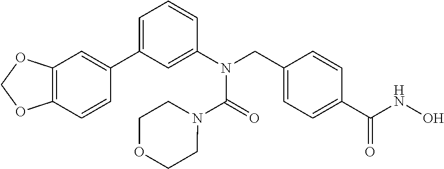 [SMILES: O=C(NO)c1ccc(CN(C(=O)N2CCOCC2)c2cccc(-c3ccc4c(c3)OCO4)c2)cc1] |
| 419 | 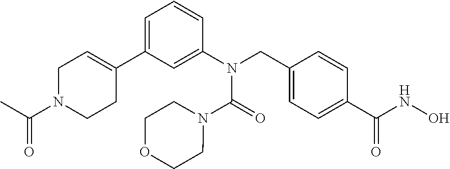 [SMILES: CC(=O)N1CC=C(c2cccc(N(Cc3ccc(C(=O)NO)cc3)C(=O)N3CCOCC3)c2)CC1] |
| 420 | 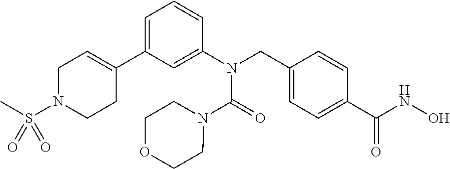 [SMILES: CS(=O)(=O)N1CC=C(c2cccc(N(Cc3ccc(C(=O)NO)cc3)C(=O)N3CCOCC3)c2)CC1] |
| 438 | 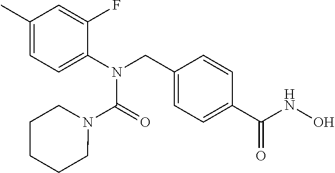 [SMILES: Cc1ccc(N(Cc2ccc(C(=O)NO)cc2)C(=O)N2CCCCC2)c(F)c1] |
| 439 | 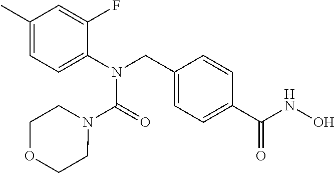 [SMILES: Cc1ccc(N(Cc2ccc(C(=O)NO)cc2)C(=O)N2CCOCC2)c(F)c1] |
| 440 | 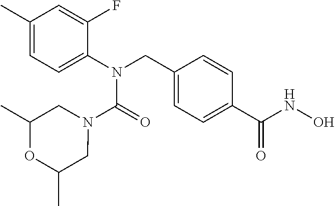 [SMILES: Cc1ccc(N(Cc2ccc(C(=O)NO)cc2)C(=O)N2CC(C)OC(C)C2)c(F)c1] |
| 441 | 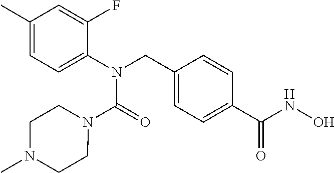 [SMILES: Cc1ccc(N(Cc2ccc(C(=O)NO)cc2)C(=O)N2CCN(C)CC2)c(F)c1] |
| 450 | 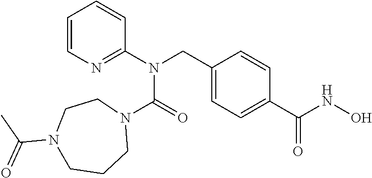 [SMILES: CC(=O)N1CCCN(C(=O)N(Cc2ccc(C(=O)NO)cc2)c2ccccn2)CC1] |
| 451 | 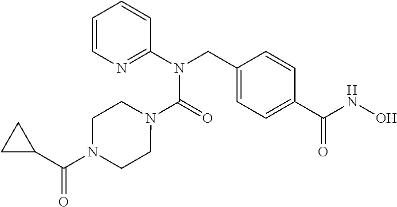 [SMILES: O=C(NO)c1ccc(CN(C(=O)N2CCN(C(=O)C3CC3)CC2)c2ccccn2)cc1] |
| 453 | 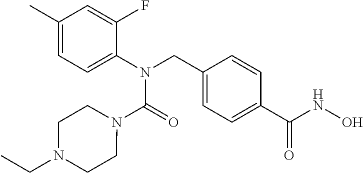 [SMILES: CCN1CCN(C(=O)N(Cc2ccc(C(=O)NO)cc2)c2ccc(C)cc2F)CC1] |
| 454 | 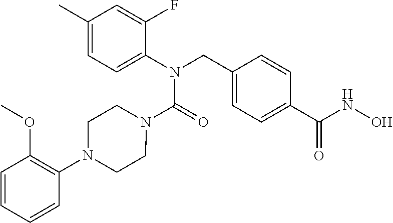 [SMILES: COc1ccccc1N1CCN(C(=O)N(Cc2ccc(C(=O)NO)cc2)c2ccc(C)cc2F)CC1] |
| 455 | 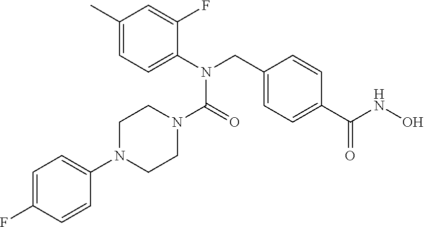 [SMILES: Cc1ccc(N(Cc2ccc(C(=O)NO)cc2)C(=O)N2CCN(c3ccc(F)cc3)CC2)c(F)c1] |

TABLE 7

| Compound | Structure |
|---|---|
| 456 | 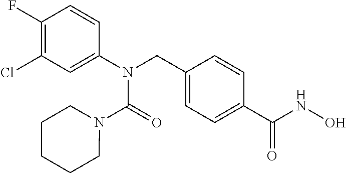 [SMILES: O=C(NO)c1ccc(CN(C(=O)N2CCCCC2)c2ccc(F)c(Cl)c2)cc1] |
| 457 | 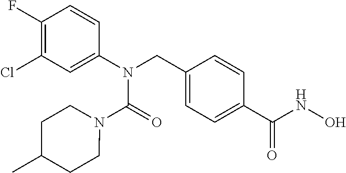 [SMILES: CC1CCN(C(=O)N(Cc2ccc(C(=O)NO)cc2)c2ccc(F)c(Cl)c2)CC1] |
| 458 | 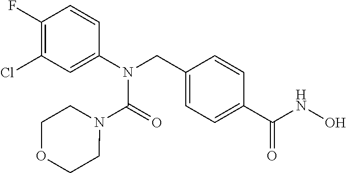 [SMILES: O=C(NO)c1ccc(CN(C(=O)N2CCOCC2)c2ccc(F)c(Cl)c2)cc1] |
| 459 | 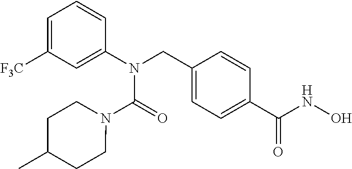 [SMILES: CC1CCN(C(=O)N(Cc2ccc(C(=O)NO)cc2)c2cccc(C(F)(F)F)c2)CC1] |
| 460 | 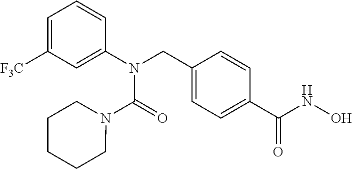 [SMILES: O=C(NO)c1ccc(CN(C(=O)N2CCCCC2)c2cccc(C(F)(F)F)c2)cc1] |
| 461 | 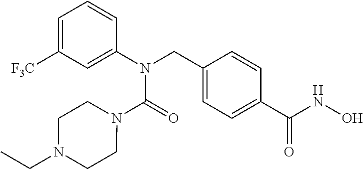 [SMILES: CCN1CCN(C(=O)N(Cc2ccc(C(=O)NO)cc2)c2cccc(C(F)(F)F)c2)CC1] |
| 462 | 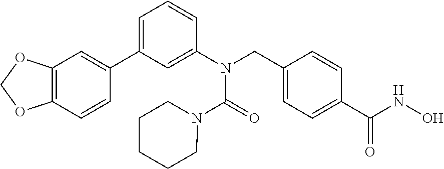 [SMILES: O=C(NO)c1ccc(CN(C(=O)N2CCCCC2)c2cccc(-c3ccc4c(c3)OCO4)c2)cc1] |
| 463 | 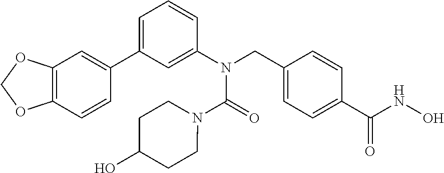 [SMILES: O=C(NO)c1ccc(CN(C(=O)N2CCC(O)CC2)c2cccc(-c3ccc4c(c3)OCO4)c2)cc1] |
| 464 | 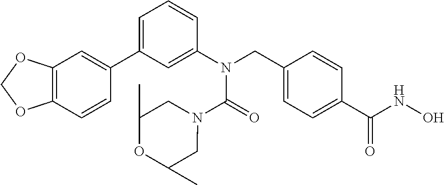 [SMILES: CC1CN(C(=O)N(Cc2ccc(C(=O)NO)cc2)c2cccc(-c3ccc4c(c3)OCO4)c2)CC(C)O1] |
| 465 | 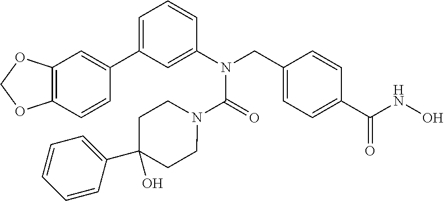 [SMILES: O=C(NO)c1ccc(CN(C(=O)N2CCC(O)(c3ccccc3)CC2)c2cccc(-c3ccc4c(c3)OCO4)c2)cc1] |
| 466 | 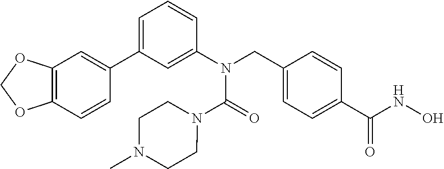 [SMILES: CN1CCN(C(=O)N(Cc2ccc(C(=O)NO)cc2)c2cccc(-c3ccc4c(c3)OCO4)c2)CC1] |
| 467 | 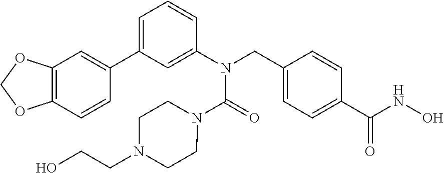 [SMILES: O=C(NO)c1ccc(CN(C(=O)N2CCN(CCO)CC2)c2cccc(-c3ccc4c(c3)OCO4)c2)cc1] |
| 468 | 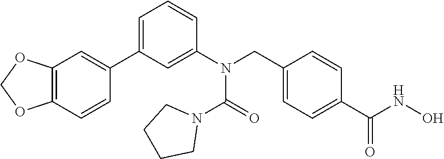 [SMILES: O=C(NO)c1ccc(CN(C(=O)N2CCCC2)c2cccc(-c3ccc4c(c3)OCO4)c2)cc1] |
| 469 | 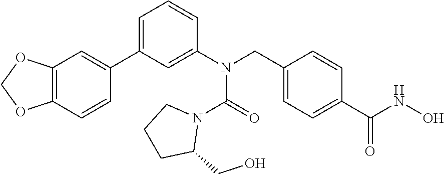 [SMILES: O=C(NO)c1ccc(CN(C(=O)N2CCC[C@H]2CO)c2cccc(-c3ccc4c(c3)OCO4)c2)cc1] |

TABLE 8

| Compound | Structure |
|---|---|
| 470 | 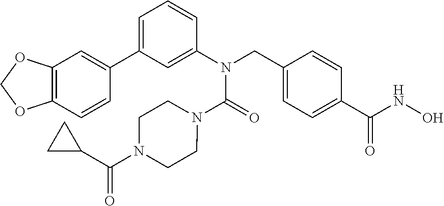 [SMILES: O=C(NO)c1ccc(CN(C(=O)N2CCN(C(=O)C3CC3)CC2)c2cccc(-c3ccc4c(c3)OCO4)c2)cc1] |
| 471 | 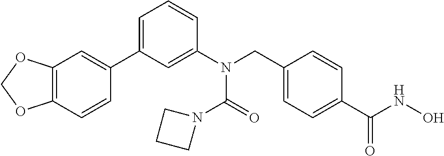 [SMILES: O=C(NO)c1ccc(CN(C(=O)N2CCC2)c2cccc(-c3ccc4c(c3)OCO4)c2)cc1] |
| 477 | 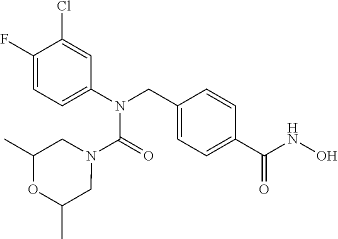 [SMILES: CC1CN(C(=O)N(Cc2ccc(C(=O)NO)cc2)c2ccc(F)c(Cl)c2)CC(C)O1] |
| 478 | 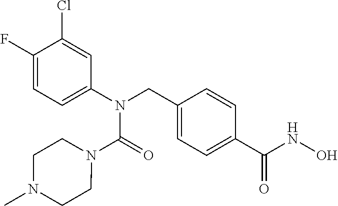 [SMILES: CN1CCN(C(=O)N(Cc2ccc(C(=O)NO)cc2)c2ccc(F)c(Cl)c2)CC1] |
| 479 | 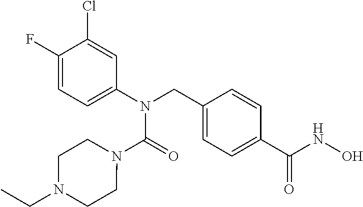 [SMILES: CCN1CCN(C(=O)N(Cc2ccc(C(=O)NO)cc2)c2ccc(F)c(Cl)c2)CC1] |
| 480 | 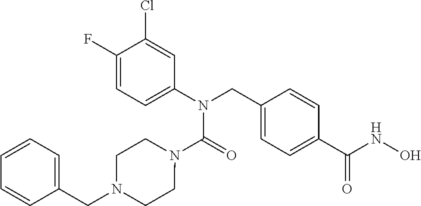 [SMILES: O=C(NO)c1ccc(CN(C(=O)N2CCN(Cc3ccccc3)CC2)c2ccc(F)c(Cl)c2)cc1] |
| 481 | 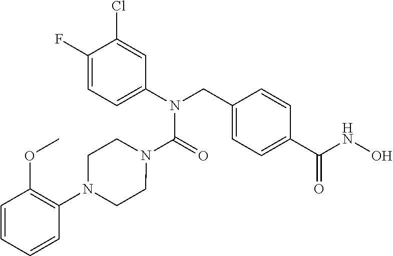 [SMILES: COc1ccccc1N1CCN(C(=O)N(Cc2ccc(C(=O)NO)cc2)c2ccc(F)c(Cl)c2)CC1] |
| 482 | 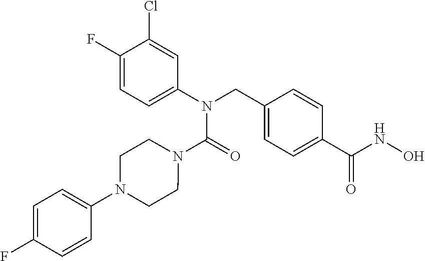 [SMILES: O=C(NO)c1ccc(CN(C(=O)N2CCN(c3ccc(F)cc3)CC2)c2ccc(F)c(Cl)c2)cc1] |
| 483 | 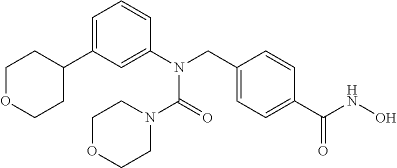 [SMILES: O=C(NO)c1ccc(CN(C(=O)N2CCOCC2)c2cccc(C3CCOCC3)c2)cc1] |
| 484 | 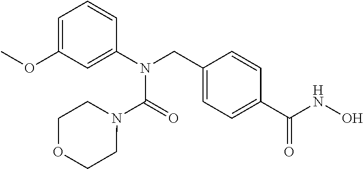 [SMILES: COc1cccc(N(Cc2ccc(C(=O)NO)cc2)C(=O)N2CCOCC2)c1] |
| 485 | 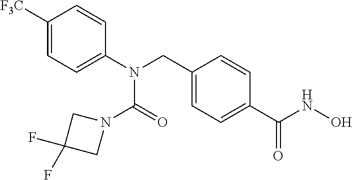 [SMILES: O=C(NO)c1ccc(CN(C(=O)N2CC(F)(F)C2)c2ccc(C(F)(F)F)cc2)cc1] |
| 486 | 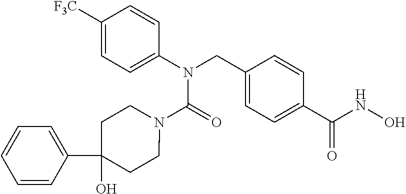 [SMILES: O=C(NO)c1ccc(CN(C(=O)N2CCC(O)(c3ccccc3)CC2)c2ccc(C(F)(F)F)cc2)cc1] |
| 487 | 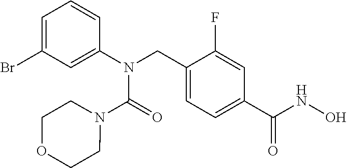 [SMILES: O=C(NO)c1ccc(CN(C(=O)N2CCOCC2)c2cccc(Br)c2)c(F)c1] |
| 488 | 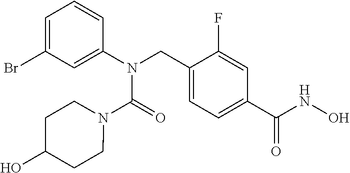 [SMILES: O=C(NO)c1ccc(CN(C(=O)N2CCC(O)CC2)c2cccc(Br)c2)c(F)c1] |

TABLE 9

| Compound | Structure |
|---|---|
| 489 | 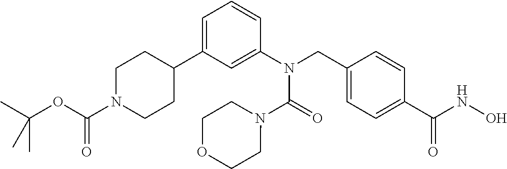 [SMILES: CC(C)(C)OC(=O)N1CCC(c2cccc(N(Cc3ccc(C(=O)NO)cc3)C(=O)N3CCOCC3)c2)CC1] |
| 490 | 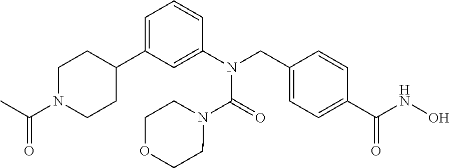 [SMILES: CC(=O)N1CCC(c2cccc(N(Cc3ccc(C(=O)NO)cc3)C(=O)N3CCOCC3)c2)CC1] |
| 491 | 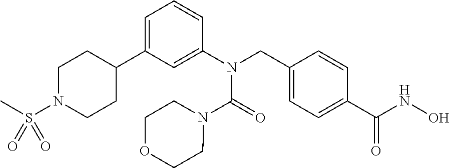 [SMILES: CS(=O)(=O)N1CCC(c2cccc(N(Cc3ccc(C(=O)NO)cc3)C(=O)N3CCOCC3)c2)CC1] |
| 492 | 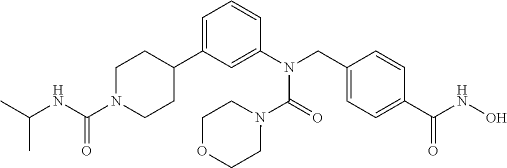 [SMILES: CC(C)NC(=O)N1CCC(c2cccc(N(Cc3ccc(C(=O)NO)cc3)C(=O)N3CCOCC3)c2)CC1] |
| 493 | 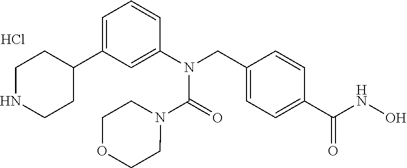 [SMILES: Cl ; O=C(NO)c1ccc(CN(C(=O)N2CCOCC2)c2cccc(C3CCNCC3)c2)cc1] |
| 494 | 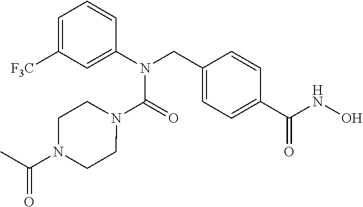 [SMILES: CC(=O)N1CCN(C(=O)N(Cc2ccc(C(=O)NO)cc2)c2cccc(C(F)(F)F)c2)CC1] |
| 495 | 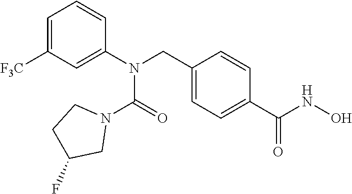 [SMILES: O=C(NO)c1ccc(CN(C(=O)N2CC[C@@H](F)C2)c2cccc(C(F)(F)F)c2)cc1] |
| 496 | 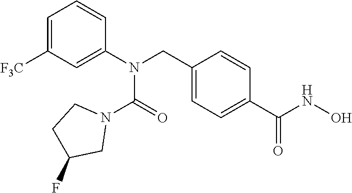 [SMILES: O=C(NO)c1ccc(CN(C(=O)N2CC[C@H](F)C2)c2cccc(C(F)(F)F)c2)cc1] |
| 497 | 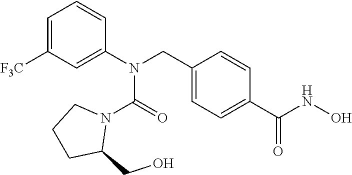 [SMILES: O=C(NO)c1ccc(CN(C(=O)N2CCC[C@@H]2CO)c2cccc(C(F)(F)F)c2)cc1] |
| 498 | 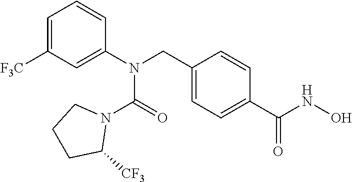 [SMILES: O=C(NO)c1ccc(CN(C(=O)N2CCC[C@H]2C(F)(F)F)c2cccc(C(F)(F)F)c2)cc1] |
| 499 | 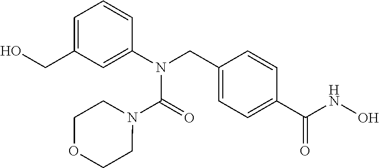 [SMILES: O=C(NO)c1ccc(CN(C(=O)N2CCOCC2)c2cccc(CO)c2)cc1] |
| 500 | 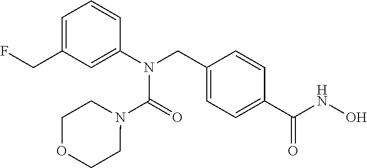 [SMILES: O=C(NO)c1ccc(CN(C(=O)N2CCOCC2)c2cccc(CF)c2)cc1] |
| 511 | 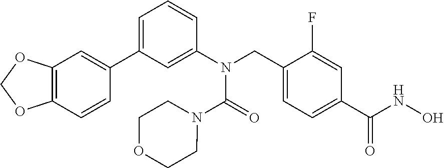 [SMILES: O=C(NO)c1ccc(CN(C(=O)N2CCOCC2)c2cccc(-c3ccc4c(c3)OCO4)c2)c(F)c1] |
| 512 | 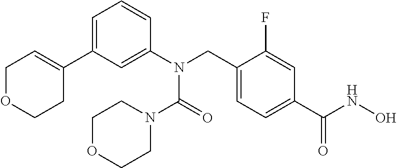 [SMILES: O=C(NO)c1ccc(CN(C(=O)N2CCOCC2)c2cccc(C3=CCOCC3)c2)c(F)c1] |

TABLE 10

| Compound | Structure |
|---|---|
| 513 | 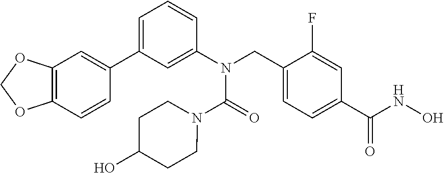 [SMILES: O=C(NO)c1ccc(CN(C(=O)N2CCC(O)CC2)c2cccc(-c3ccc4c(c3)OCO4)c2)c(F)c1] |
| 514 | 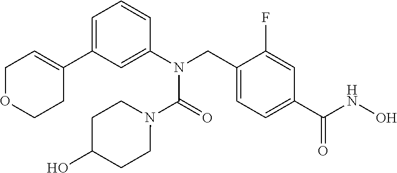 [SMILES: O=C(NO)c1ccc(CN(C(=O)N2CCC(O)CC2)c2cccc(C3=CCOCC3)c2)c(F)c1] |
| 517 | 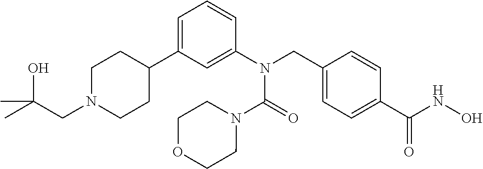 [SMILES: CC(C)(O)CN1CCC(c2cccc(N(Cc3ccc(C(=O)NO)cc3)C(=O)N3CCOCC3)c2)CC1] |
| 518 | 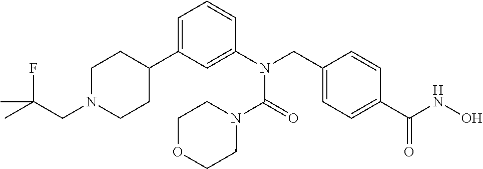 [SMILES: CC(C)(F)CN1CCC(c2cccc(N(Cc3ccc(C(=O)NO)cc3)C(=O)N3CCOCC3)c2)CC1] |
| 520 | 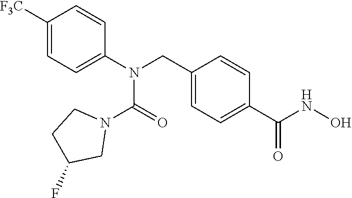 [SMILES: O=C(NO)c1ccc(CN(C(=O)N2CC[C@@H](F)C2)c2ccc(C(F)(F)F)cc2)cc1] |
| 521 | 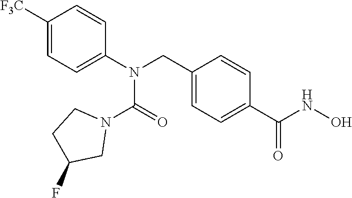 [SMILES: O=C(NO)c1ccc(CN(C(=O)N2CC[C@H](F)C2)c2ccc(C(F)(F)F)cc2)cc1] |
| 522 | 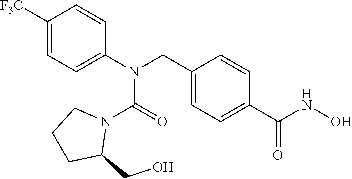 [SMILES: O=C(NO)c1ccc(CN(C(=O)N2CCC[C@@H]2CO)c2ccc(C(F)(F)F)cc2)cc1] |
| 529 | 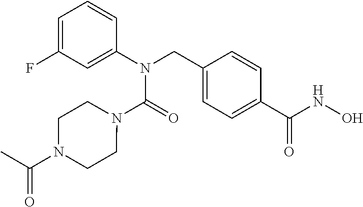 [SMILES: CC(=O)N1CCN(C(=O)N(Cc2ccc(C(=O)NO)cc2)c2cccc(F)c2)CC1] |
| 530 | 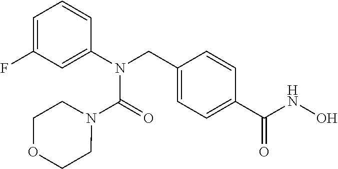 [SMILES: O=C(NO)c1ccc(CN(C(=O)N2CCOCC2)c2cccc(F)c2)cc1] |
| 531 | 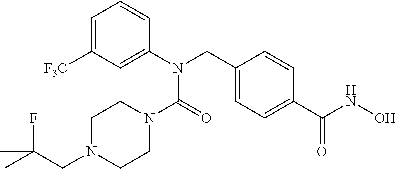 [SMILES: CC(C)(F)CN1CCN(C(=O)N(Cc2ccc(C(=O)NO)cc2)c2cccc(C(F)(F)F)c2)CC1] |
| 532 | 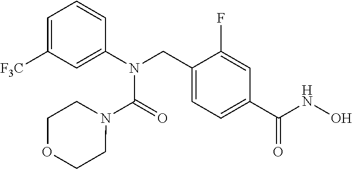 [SMILES: O=C(NO)c1ccc(CN(C(=O)N2CCOCC2)c2cccc(C(F)(F)F)c2)c(F)c1] |
| 533 | 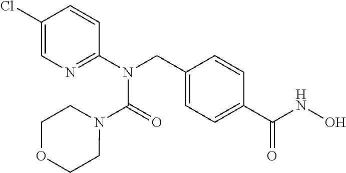 [SMILES: O=C(NO)c1ccc(CN(C(=O)N2CCOCC2)c2ccc(Cl)cn2)cc1] |
| 543 | 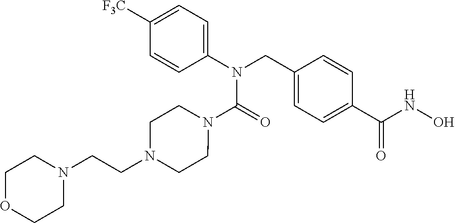 [SMILES: O=C(NO)c1ccc(CN(C(=O)N2CCN(CCN3CCOCC3)CC2)c2ccc(C(F)(F)F)cc2)cc1] |
| 544 | 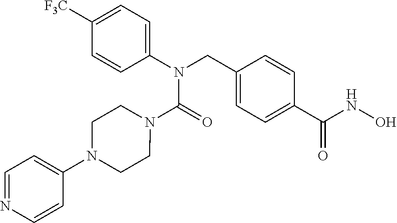 [SMILES: O=C(NO)c1ccc(CN(C(=O)N2CCN(c3ccncc3)CC2)c2ccc(C(F)(F)F)cc2)cc1] |

TABLE 11

| Compound | Structure |
|---|---|
| 545 | 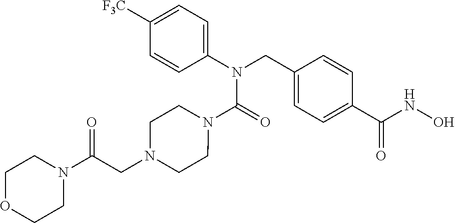 [SMILES: O=C(NO)c1ccc(CN(C(=O)N2CCN(CC(=O)N3CCOCC3)CC2)c2ccc(C(F)(F)F)cc2)cc1] |
| 577 | 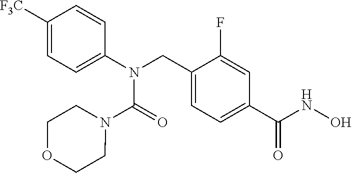 [SMILES: O=C(NO)c1ccc(CN(C(=O)N2CCOCC2)c2ccc(C(F)(F)F)cc2)c(F)c1] |
| 578 | 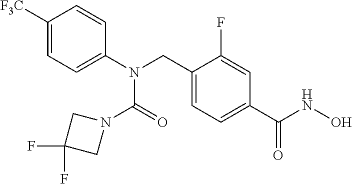 [SMILES: O=C(NO)c1ccc(CN(C(=O)N2CC(F)(F)C2)c2ccc(C(F)(F)F)cc2)c(F)c1] |
| 580 | 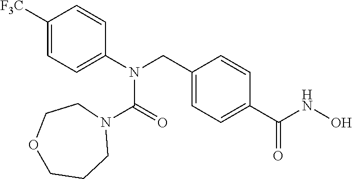 [SMILES: O=C(NO)c1ccc(CN(C(=O)N2CCCOCC2)c2ccc(C(F)(F)F)cc2)cc1] |
| 651 | 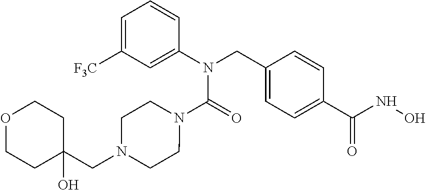 [SMILES: O=C(NO)c1ccc(CN(C(=O)N2CCN(CC3(O)CCOCC3)CC2)c2cccc(C(F)(F)F)c2)cc1] |
| 683 | 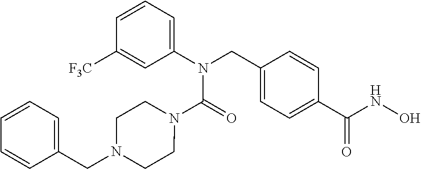 [SMILES: O=C(NO)c1ccc(CN(C(=O)N2CCN(Cc3ccccc3)CC2)c2cccc(C(F)(F)F)c2)cc1] |
| 684 | 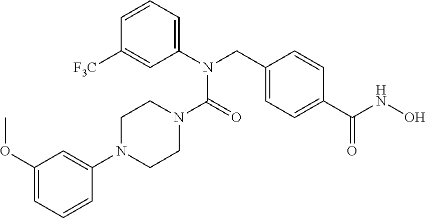 [SMILES: COc1cccc(N2CCN(C(=O)N(Cc3ccc(C(=O)NO)cc3)c3cccc(C(F)(F)F)c3)CC2)c1] |
| 716 | 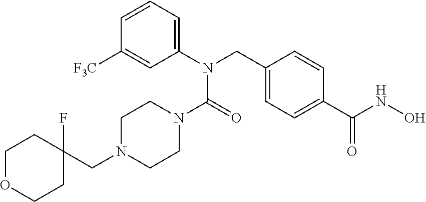 [SMILES: O=C(NO)c1ccc(CN(C(=O)N2CCN(CC3(F)CCOCC3)CC2)c2cccc(C(F)(F)F)c2)cc1] |
| 717 | 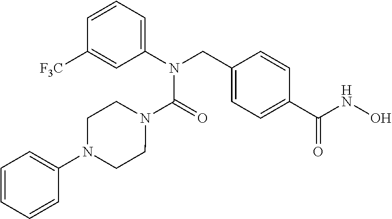 [SMILES: O=C(NO)c1ccc(CN(C(=O)N2CCN(c3ccccc3)CC2)c2cccc(C(F)(F)F)c2)cc1] |
| 718 | 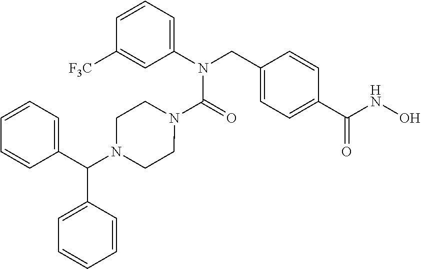 [SMILES: O=C(NO)c1ccc(CN(C(=O)N2CCN(C(c3ccccc3)c3ccccc3)CC2)c2cccc(C(F)(F)F)c2)cc1] |
| 765 | 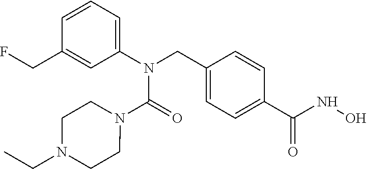 [SMILES: CCN1CCN(C(=O)N(Cc2ccc(C(=O)NO)cc2)c2cccc(CF)c2)CC1] |
| 766 | 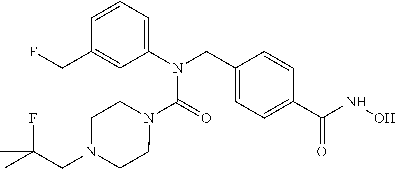 [SMILES: CC(C)(F)CN1CCN(C(=O)N(Cc2ccc(C(=O)NO)cc2)c2cccc(CF)c2)CC1] |
| 771 | 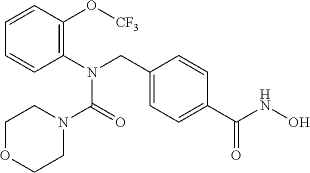 [SMILES: O=C(NO)c1ccc(CN(C(=O)N2CCOCC2)c2ccccc2OC(F)(F)F)cc1] |
| 772 | 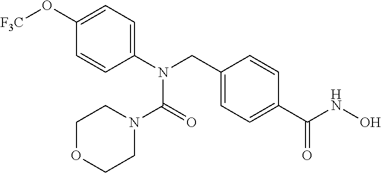 [SMILES: O=C(NO)c1ccc(CN(C(=O)N2CCOCC2)c2ccc(OC(F)(F)F)cc2)cc1] |

TABLE 12

| Compound | Structure |
|---|---|
| 773 | 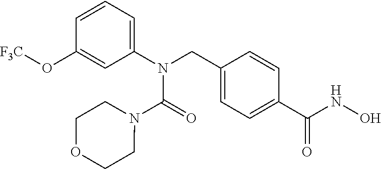 [SMILES: O=C(NO)c1ccc(CN(C(=O)N2CCOCC2)c2cccc(OC(F)(F)F)c2)cc1] |
| 774 | 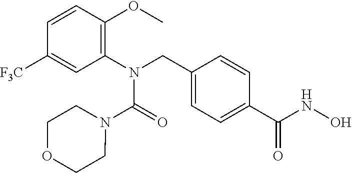 [SMILES: COc1ccc(C(F)(F)F)cc1N(Cc1ccc(C(=O)NO)cc1)C(=O)N1CCOCC1] |
| 776 | 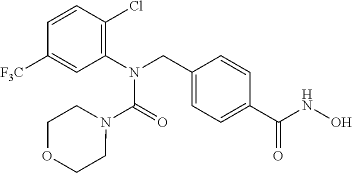 [SMILES: O=C(NO)c1ccc(CN(C(=O)N2CCOCC2)c2cc(C(F)(F)F)ccc2Cl)cc1] |
| 778 | 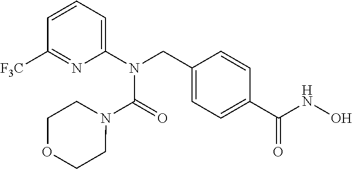 [SMILES: O=C(NO)c1ccc(CN(C(=O)N2CCOCC2)c2cccc(C(F)(F)F)n2)cc1] |
| 791 | 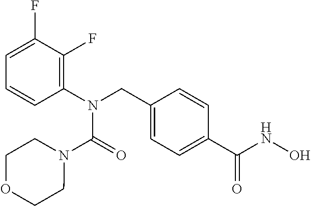 [SMILES: O=C(NO)c1ccc(CN(C(=O)N2CCOCC2)c2cccc(F)c2F)cc1] |
| 797 | 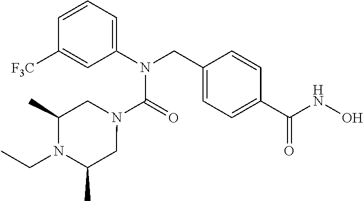 [SMILES: CCN1[C@H](C)CN(C(=O)N(Cc2ccc(C(=O)NO)cc2)c2cccc(C(F)(F)F)c2)C[C@@H]1C] |
| 800 | 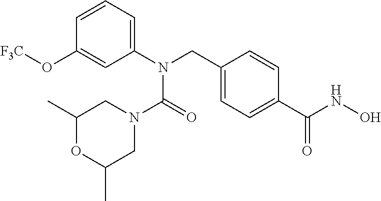 [SMILES: CC1CN(C(=O)N(Cc2ccc(C(=O)NO)cc2)c2cccc(OC(F)(F)F)c2)CC(C)O1] |
| 801 | 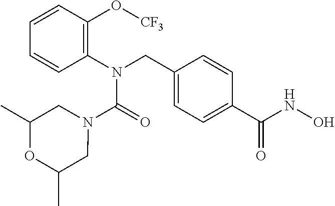 [SMILES: CC1CN(C(=O)N(Cc2ccc(C(=O)NO)cc2)c2ccccc2OC(F)(F)F)CC(C)O1] |
| 802 | 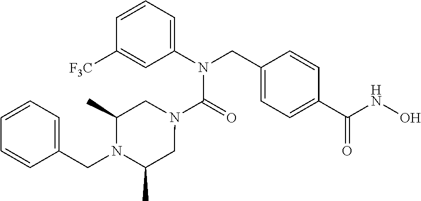 [SMILES: C[C@@H]1CN(C(=O)N(Cc2ccc(C(=O)NO)cc2)c2cccc(C(F)(F)F)c2)C[C@H](C)N1Cc1ccccc1] |
| 803 | 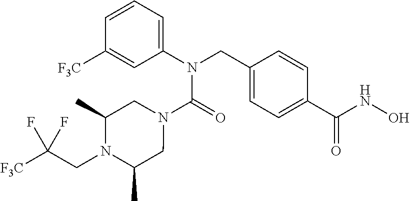 [SMILES: C[C@@H]1CN(C(=O)N(Cc2ccc(C(=O)NO)cc2)c2cccc(C(F)(F)F)c2)C[C@H](C)N1CC(F)(F)C(F)(F)F] |
| 826 | 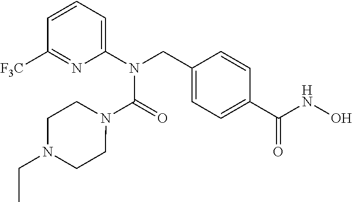 [SMILES: CCN1CCN(C(=O)N(Cc2ccc(C(=O)NO)cc2)c2cccc(C(F)(F)F)n2)CC1] |
| 827 | 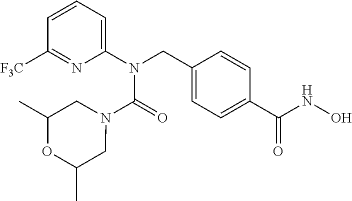 [SMILES: CC1CN(C(=O)N(Cc2ccc(C(=O)NO)cc2)c2cccc(C(F)(F)F)n2)CC(C)O1] |
| 828 | 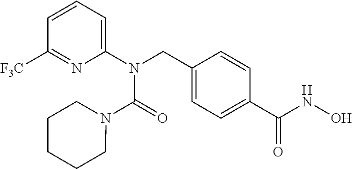 [SMILES: O=C(NO)c1ccc(CN(C(=O)N2CCCCC2)c2cccc(C(F)(F)F)n2)cc1] |
| 829 | 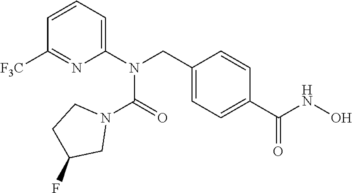 [SMILES: O=C(NO)c1ccc(CN(C(=O)N2CC[C@H](F)C2)c2cccc(C(F)(F)F)n2)cc1] |

In the present invention, the compound represented by the formula I above may be prepared by means of a method disclosed in Korea Unexamined Patent Application Publication No. 2014-0128886, but is not limited thereto.

In the present invention, a pharmaceutically acceptable salt means a salt conventionally used in an industry of medicine, e.g., an inorganic ion salt prepared from calcium, potassium, sodium, magnesium and the like; an inorganic acid salt prepared from hydrochloric acid, nitric acid, phosphoric acid, bromic acid, iodic acid, perchloric acid, sulfuric acid and the like; an organic acid salt prepared from acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbric acid, carbonic acid, vanillic acid, hydroiodic acid, etc.; a sulphonic acid salt prepared from methanesulfonic acid, ethanesulfonic acid, benzenesulfonic acid, p-toluenesulfonic acid, naphthalenesulfonic acid and the like; amino acid salt prepared from glycine, arginine, lysine, etc.; amine salt prepared from trimethylamine, triethylamine, ammonia, pyridine, picoline, etc.; and the like, but the types of salt meant in the present invention are not limited to those listed salts.

As used herein, the term “dry eye” means a tear film disease, which causes irritation to an eye, for example, an eye disease, which occurs due to a lack of tear, an excessive evaporation of tear or an imbalance in tear components, and shows dry eye symptoms such as feeling of irritation caused by foreign matter, burning sensation, itchiness, irritation, redness, eye pain, blurred vision, loss of vision and excessive tear secretion. In the present invention, dry eye may be a result of other underlying diseases such as Sjogren's syndrome, and may be a complication of inflammation, i.e., ophthalmodesmitis, or a foreign matter inside an eye. Also, in the present invention, dry eye may be a result of infection or a side effect of medication, and an exposure to toxin, chemicals or other substances may become a cause for dry eye symptom or disease. In other words, in the present invention, dry eye may include both dry eye caused by Sjogren's syndrome and dry eye caused by non-Sjogren's syndrome.

In the present invention, dry eye may be prevented or treated by means of an administration of a pharmaceutical composition according to the present invention. For example, the pharmaceutical composition according to the present invention may prevent or treat dry eye through the mediation of immunoregulation. In one embodiment of the present invention, the said immunoregulation may be to inhibit an expression of inflammatory cytokines.

In an example of the present invention, it was identified that a pharmaceutical composition containing a compound represented by Formula I, an optical isomer thereof or a pharmaceutically acceptable salt thereof may not only improve a corneal erosion in an animal model of induced dry eye (FIGS. 1 and 2), but also effectively inhibit an expression of inflammatory cytokines involved in dry eye (FIG. 3), thus having an excellent effect on treatment of dry eye.

A pharmaceutical composition according to the present invention may further comprise at least one type of a pharmaceutically acceptable carrier, in addition to the compound represented by the formula I above, the optical isomer thereof or the pharmaceutically acceptable salt thereof, for the purpose of administration. As the pharmaceutically acceptable carrier, saline solution, sterilized water, Ringer's solution, buffered saline, dextrose solution, maltodextrin solution, glycerol, ethanol and a combination of at least one component thereof may be used, wherein other conventional additives such as antioxidant, buffer solution, bacteriostatic agent, etc., may be also added thereto, if needed. Also, the pharmaceutical composition according to the present invention may be formulated into an injectable dosage form such as aqueous solution, suspension, emulsion, etc., pill, capsule, granule or tablet in such a way that diluent, dispersing agent, surfactant, binder and lubricant are further added thereto. Thus, the composition according to the present invention may be a patch, liquid medicine, pill, capsule, granule, tablet, suppository, etc. These preparations may be formulated by means of a conventional method used for formulation in the technical field to which the present invention pertains according to each disease and/or component, or a method disclosed in Remington's Pharmaceutical Science (the latest version), Mack Publishing Company, Easton, Pa.

A non-limiting example of a preparation for oral administration using the pharmaceutical composition according to the present invention may be a tablet, troche, lozenge, water soluble suspension, oil suspension, prepared powder, granule, emulsion, hard capsule, soft capsule, syrup, elixir or the like. To formulate the pharmaceutical composition according to the present invention into a preparation for oral administration, a binder such as lactose, saccharose, sorbitol, mannitol, starch, amylopectin, cellulose, gelatin or the like; an excipient such as dicalcium phosphate, etc.; a disintegrant such as maize starch, sweet potato starch or the like; a lubricant such as magnesium stearate, calcium stearate, sodium stearyl fumarate, polyethylene glycol wax or the like; and so on may be used, and a sweetening agent, flavoring agent, syrup, etc., may be also used. Furthermore, in case of the capsule, a liquid carrier such as fatty oil, etc. may be further used in addition to the above-mentioned materials.

A non-limiting example of a parenteral preparation using the pharmaceutical composition according to the present invention may be an injectable solution, suppository, powder for respiratory inhalation, aerosol preparation for spray, ointment, powder for application, oil, cream, etc. To formulate the pharmaceutical composition according to the present invention into a preparation for parenteral administration, a sterilized aqueous solution, nonaqueous solvent, suspension, emulsion, freeze-dried preparation, external preparation, etc. may be used. As the said nonaqueous solvent and suspension, a vegetable oil such as propylene glycol, polyethylene glycol and olive oil; an injectable ester such as ethyl oleate; and so on may be used, for example, an ophthalmic solution or emulsion, an ophthalmic gel, an ophthalmic ointment or an oily lotion, which contains a composition for eye drop, may be used, but not limited thereto.

The pharmaceutical composition of the present invention may be orally or parenterally administered. In case of the parental administration, such composition may be locally administered, for example, through eye drops, but not limited thereto.

If the pharmaceutical composition according to the present invention is used in a form of a composition for eye drop, the said composition for eye drop may be prepared by suspending a compound of Formula I according to the present invention, an optical isomer thereof or a pharmaceutically acceptable salt thereof in sterile aqueous solution, for example, salt water, buffer solution, etc., or by compounding the above-mentioned compositions in a form of soluble powder therein before use. Other additives, for example, an isotonic agent (e.g. sodium chloride, etc.), a buffer agent (e.g. boric acid, sodium monohydrogen phosphate, sodium dihydrogen phosphate, etc.), a preservative agent (e.g. benzalkonium chloride, benzethonium chloride, chlorobutanol, etc.), a thickening agent (e.g. sugars, for example, lactose, mannitol, maltose, etc.; e.g. hyaluronic acid or salt thereof, for example, sodium hyaluronate, potassium hyaluronate, etc.; e.g. mucopolysaccharides, for example, chondroitin sulfate, etc.; e.g. sodium polyacrylate, a carboxy vinyl polymer, cross-linked polyacrylic acid salt, etc.) may be included in the composition for eye drop.

In an example of the present invention, it was identified that the pharmaceutical composition containing a compound represented by Formula I, an optical isomer thereof or a pharmaceutically acceptable salt thereof may not only improve a corneal erosion in an animal model of induced dry eye, but also effectively inhibit an expression of inflammatory cytokines involved in dry eye, thus having an excellent effect on treatment of dry eye.

A daily dosage of a compound represented by a formula I according to the present invention, an optical isomer thereof or a pharmaceutically acceptable salt thereof may fall, for example, in a range of about 0.1 to 10,000 mg/kg, in a range of about 1 to 8,000 mg/kg, in a range of about 5 to 6,000 mg/kg, or in a range of about 10 to 4,000 mg/kg, preferably in a range of about 50 to 2,000 mg/kg, but is not limited thereto, wherein such dosage may be also administered once a day or divided into several times a day for administration.

A pharmaceutically effective amount and effective dosage of the pharmaceutical composition according to the present invention may be diversified by means of a method for formulating the pharmaceutical composition into a preparation, an administration mode, an administration time and/or administration route, etc., and may be diversified according to various factors including a type and degree of reactions to be achieved by means of an administration of the pharmaceutical composition, a type of an individual to be administered, age, weight, general health condition, a symptom or severity of disease, gender, diet, excretion, a component of other drug compositions used together at the same time or different times for the corresponding individual, and so on, as well as other similar factors well known in a field of medicine, wherein those skilled in the art may easily determine and prescribe a dosage effective for targeted treatment.

In case of the administration of the pharmaceutical composition according to the present invention, it may be administered once a day or divided into several times a day for administration. The pharmaceutical composition according to the present invention may be administered as an individual therapeutic agent or in combination with other therapeutic agents, and may be also administered sequentially or simultaneously with a conventional therapeutic agent. Considering all the factors above, the pharmaceutical composition according to the present invention may be administered by an amount, which can show the maximum effect with the minimum amount without any side effect, wherein such amount may be easily determined by those skilled in the art to which the present invention pertains.

The pharmaceutical composition according to the present invention may show an excellent effect even when used alone, but may be further used in combination with various methods such as hormone therapy, drug treatment, etc. so as to increase a therapeutic efficiency.

The present invention also provides a method for treating dry eye, wherein the method comprises administering a therapeutically effective amount of the compound represented by the formula I above, the optical isomer thereof or the pharmaceutically acceptable salt thereof into an individual in need.

As used herein, the term “therapeutically effective amount” refers to an amount of the compound represented by the formula I above, the optical isomer thereof or the pharmaceutically acceptable salt thereof which is effective in treating dry eye.

In the treatment method according to the present invention, a suitable total daily dose of the compound represented by the formula I above, the optical isomer thereof or the pharmaceutically acceptable salt thereof may be determined by a doctor in charge within the range of correct medical decision, and may fall, for example, in a range of about 0.1 to 10,000 mg/kg, in a range of about 1 to 8,000 mg/kg, in a range of about 5 to 6,000 mg/kg, or in a range of about 10 to 4,000 mg/kg, and preferably such dose in a range of about 50 to 2,000 mg/kg may be administered once a day or divided into several times a day for administration. However, for the purpose of the present invention, it is preferable that a specific, therapeutically effective amount for a certain patient is differently applied depending on various factors including a type and degree of reactions to be achieved, a specific composition including whether other preparations are used or not in some cases, a patient's age, weight, general health condition, gender and diet, an administration time, an administration route and a secretion rate of the composition, a treatment period, and a drug used together or simultaneously with the specific composition, as well as other similar factors well known in a field of medicine.

The method for treating dry eye according to the present invention comprises not only dealing with the disease itself before expression of its symptoms, but also inhibiting or avoiding such symptoms by administering the compound represented by the formula I above, the optical isomer thereof or the pharmaceutically acceptable salt thereof. In managing the disease, a preventive or therapeutic dose of a certain active component may vary depending on characteristics and severity of the disease or condition, and a route in which the active component is administered. The dose and a frequency thereof may vary depending on an individual patient's age, weight and reactions. A suitable dose and usage may be easily selected by those skilled in the art, naturally considering such factors. Also, the method for treating dry eye according to the present invention may further comprise administering a therapeutically effective dose of an additional active agent, which is helpful in treating the disease, along with the compound represented by the formula I above, the optical isomer thereof or the pharmaceutically acceptable salt thereof wherein the additional active agent may show a synergy effect or an additive effect together with the compound of the formula I above, the optical isomer thereof or the pharmaceutically acceptable salt thereof.

The present invention also provides a use of the compound represented by the formula I above, the optical isomer thereof or the pharmaceutically acceptable salt thereof in the manufacture of a medicament for treating dry eye. The compound represented by the formula I above, the optical isomer thereof or the pharmaceutically acceptable salt thereof for the manufacture of a medicament may be combined with a pharmaceutically acceptable adjuvant, diluent, carrier, etc., and may be prepared into a composite agent together with other active agents, thus having a synergy action.

The matters mentioned in the inventive pharmaceutical composition, the treatment method and the use are equally applied, if not contradictory to each other.

### Advantageous Effects of Invention

A pharmaceutical composition comprising a compound represented by a formula I according to the present invention, an optical isomer thereof or a pharmaceutically acceptable salt thereof may show an excellent effect of treating dry eye, such that the pharmaceutical composition may be widely used for prevention or treatment of dry eye.

### BRIEF DESCRIPTION OF DRAWINGS

FIG. 1 shows a result of performing a corneal erosion test in a mouse of induced dry eye.

FIG. 2 shows a result of staining a cornea in the mouse of induced dry eye.

FIG. 3 shows an expression level of cytokines in the mouse of inflammatory dry eye.

### MODE FOR THE INVENTION

Hereinafter, the present invention will be described in more detail according to preparation examples and embodiments. However, these preparation examples and embodiments are provided only for the purpose of illustrating the present invention, and thus the present invention is not limited thereto.

### Preparation Example 1. Synthesis of {Compound 374} {N-(4-(hydroxycarbamoyl)benzyl)-N-(3-(trifluoromethyl)phenyl)morpholine-4-carboxamide}

[Step 1] Synthesis of Methyl 4-((3-(trifluoromethyl)phenylamino)methyl)benzoate

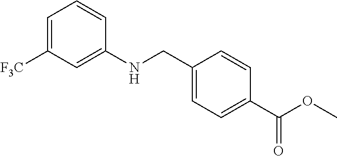 [SMILES: COC(=O)c1ccc(CNc2cccc(C(F)(F)F)c2)cc1]

3-(trifluoromethyl)benzeneamine (0.30 g, 1.84 mmol) and potassium carbonate (0.76 g, 5.53 mmol) were dissolved in dimethylformamide (DMF) (5 mL), and then methyl 4-(bromomethyl)benzoate (0.42 g, 1.84 mmol) was inserted thereinto. A resulting mixture was reacted at room temperature for a day and diluted with ethyl acetate. A reactant was washed with water and saturated sodium chloride aqueous solution, then dried by means of anhydrous magnesium sulfate and filtered, and then concentrated under reduced pressure. A residue was purified via column chromatography (silicon dioxide; ethyl acetate/hexane=20%), such that a title compound (0.37 g, 65%) was obtained.

¹H NMR (400 MHz, DMSO-d₆) δ 7.93 (d, 2 H, J=8.3 Hz), 7.49 (d, 2 H, J=8.3 Hz), 7.24 (t, 1 H, J=7.9 Hz), 6.88-6.78 (m, 4 H), 4.42 (d, 2 H, J=6.1 Hz), 3.83 (s, 3H), MS (ESI) m/z 310 (M⁺+H).

[Step 2] Synthesis of Methyl 4-((((4-nitrophenoxy)carbonyl)(3-(trifluoromethyl)phenyl)amino)methyl)benzoate

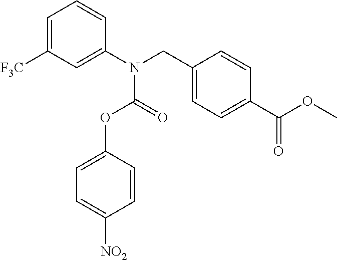 [SMILES: COC(=O)c1ccc(CN(C(=O)Oc2ccc([N+](=O)[O-])cc2)c2cccc(C(F)(F)F)c2)cc1]

Methyl 4-((3-(trifluoromethyl)phenylamino)methyl)benzoate (0.26 g, 0.82 mmol) and 4-nitrophenyl carbonochloridate (0.33 g, 1.65 mmol) were dissolved in acetonitrile (10 mL), and then potassium carbonate (0.34 g, 2.47 mmol) was inserted thereinto. A resulting mixture was reacted at room temperature for a day and diluted with ethyl acetate. A reactant was washed with saturated sodium chloride aqueous solution, then dried by means of anhydrous sodium sulfate and filtered, and then concentrated under reduced pressure. A residue was purified via column chromatography (silicon dioxide; ethyl acetate/hexane=20%), such that a title compound (0.35 g, 89%) was obtained in a colorless oil form.

¹H NMR (400 MHz, CDCl₃) δ 8.20 (d, 2 H, J=10.2 Hz), 8.01 (d, 2 H, J=7.8 Hz), 7.56-7.46 (m, 3H), 7.35 (d, 3 H, J=8.0 Hz), 7.26 (d, 2 H, J=8.1 Hz), 5.01 (bs, 2H), 3.90 (s, 3H).

[Step 3] Synthesis of Methyl 4-((N-(3-(trifluoromethyl)phenyl)morpholine-4-carboxamido)methyl)benzoate

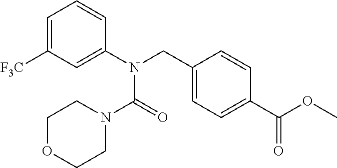 [SMILES: COC(=O)c1ccc(CN(C(=O)N2CCOCC2)c2cccc(C(F)(F)F)c2)cc1]

Methyl

4-((((4-nitrophenoxy)carbonyl)(3-(trifluoromethyl)phenyl)amino)methyl)benzoate (0.29 g, 0.60 mmol) was dissolved in dimethylformamide (10 ml), and then potassium carbonate (0.25 g, 1.81 mmol) and morpholine (0.05 mL, 0.60 mmol) were inserted thereinto. A resulting mixture was reacted at 60° C. for two days, and then diluted with saturated ammonium chloride solution. Extraction was performed by means of ethyl acetate, and then an extract was dried by means of anhydrous sodium sulfate and filtered, and then concentrated under reduced pressure. A residue was purified via column chromatography (silicon dioxide; ethyl acetate/hexane=50%), such that a title compound (0.15 g, 60%) was obtained.

¹H NMR (400 MHz, DMSO-d₆) δ 7.97 (d, 2 H, J=8.2 Hz), 7.43-7.32 (m, 5H), 7.20 (d, 1 H, J=8.0 Hz), 4.94 (s, 2H), 3.90 (s, 3H), 3.50 (t, 4 H, J=4.8 Hz), 3.25 (t, 4 H, J=4.8 Hz); MS (ESI) m/z 423 (M⁺+H).

[Step 4] Synthesis of N-(4-(hydroxycarbamoyl) benzyl)-N-(3-(trifluoromethyl)phenyl)morpholine-4-carboxamide

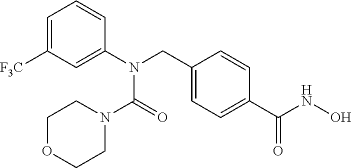 [SMILES: O=C(NO)c1ccc(CN(C(=O)N2CCOCC2)c2cccc(C(F)(F)F)c2)cc1]

Methyl

4-((N-(3-(trifluoromethyl)phenyl)morpholine-4-carboxamido)methyl)benzoate (0.15 g, 0.36 mmol) was dissolved in methanol (5 mL), then hydroxylamine aqueous solution (50 wt%, 1 mL) and potassium hydroxide (0.10 g, 1.81 mmol) were inserted thereinto, and then stirred overnight. After a reaction was completed, methanol was distilled under reduced pressure and removed, and then extraction was performed by means of ethyl acetate and water, and then worked up. A resulting extract was dried by means of anhydrous sodium sulfate and filtered, and then concentrated under reduced pressure. A residue was stirred in diethyl ether, and then a solid product was made, filtered and dried, such that a title compound (0.082 g, 54%) was obtained in a white solid form.

¹H NMR (400 MHz, MeOD-d₃) δ 11.14 (brs, 1 H), 8.99 (brs, 1 H), 7.85 (d, 2 H, J =8.0 Hz), 7.66-7.27 (m, 6 H), 4.94 (s, 2 H), 3.41 (s, 2 H), 3.15 (s, 2 H). MS (ESI) m/z 424 (M⁺+H).

### Preparation Example 2. Establishment of an Animal Model of Induced Dry Eye

Everyday, 0.5 mg/0.2 mL of scopolamine hydrobromide was subcutaneously injected into a C57BL/6 mouse, which was then subjected to air ventilation for 18 hours everyday in an environment with humidity of 40% or less. In this way, the mouse was kept for seven days, thereby establishing a mouse model of induced dry eye.

With regard to the said mouse model, a compound 374 of the present invention, prepared in Preparation Example 1, was dissolved 0.1% in 10% ethanol (vehicle-1) or 5% polyethylene glycol 300 (vehicle-2) solution, and then a resulting mixture was administered through eye drops into an eye of the dry eye mouse once daily for seven days (Table 13).

TABLE 13

| Group | | Administration | Number of |
|---|---|---|---|
| name | Concentration | route | administrations |
| Normal | — | — | — |
| (Control) | | | |
| Vehicle-1 | — | Eye drop | Once/day |
| Vehicle-2 | — | Eye drop | Once/day |
| Compound | 0.1% | Eye drop | Once/day |
| 374 + | | | |
| Vehicle-1 | | | |
| (374-1) | | | |
| Compound | 0.1% | Eye drop | Once/day |
| 374 + | | | |
| Vehicle-2 | | | |
| (374-2) | | | |

### Example 1. Identification of an Improvement Effect on Corneal Erosion in a Dry Eye Mouse

To identify a therapeutic effect of the compound according to the present invention on dry eye, a corneal staining was performed with a fluorescent dye on a cornea of the mouse of induced dry eye, and then a corneal erosion grade was evaluated by means of an Oxford scheme while checking the same with a slit lamp biomicroscopy (Bron A J, Evans V E, Smith J A. Grading of corneal and conjunctival staining in the context of other dry eye tests. Cornea. 2003; 22(7):640-50, Grade 0: normal, Grade 5: most severe corneal damage). (** p=0.036. p values were calculated by means of the Kruskall-Wallis method based on Dunn's multiple comparison test as post hoc test.)

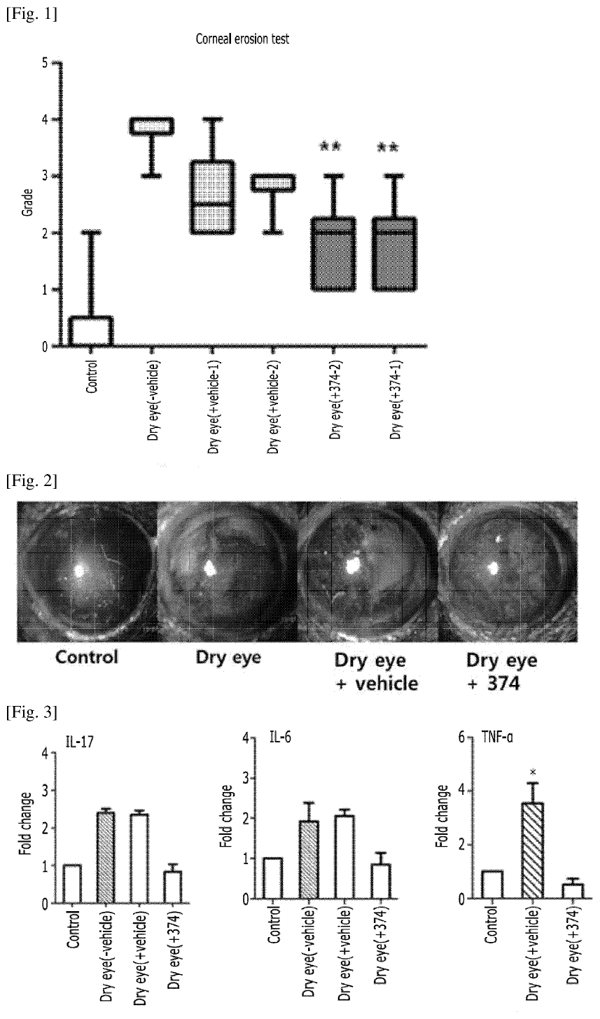

As a result, it was identified that the mouse of induced dry eye (no vehicle, vehicle-1 and vehicle-2) shows a deterioration of corneal erosion symptom up to Grade 4, but the experimental group of mice dosed with the compound of the present invention through eye drops shows a remarkable improvement in the corneal erosion grade to a level of Grades 1 to 2 regardless of the solvent (vehicle-1 and vehicle-2) (FIG. 1). As a result of observing an eyeball of the mouse with naked eyes, it was identified that a considerable corneal damage occurs to the mouse of induced dry eye dosed with the vehicle-1 through eye drops or not, but a degree of corneal damage is remarkably alleviated in the mouse dosed with the compound of the present invention through eye drops (FIG. 2).

The results above suggest that the compound of the present invention may be effectively used in treatment of dry eye.

### Example 2. Inhibitory Effect on Expression of Inflammatory Cytokines in the Dry Eye Mouse

To identify if a value of inflammatory cytokines, known to show an increased expression in dry eye, is inhibited by means of an administration of the compound of the present invention, a real-time PCR was performed on a corneal tissue of the mouse of induced dry eye.

To perform the real-time PCR, a cornea was separated from a pre-extracted eyeball of the dry eye mouse, and then cells thereof were dissolved with a trizol reagent. Then, with regard to an RNA sample, cDNA was synthesized by using PrimeSctipt RT Master (TAKARA). After that, the real-time PCR was performed with StepOne-PlusReal-Time PCR System (Applied Biosystems) by using SYBR Premix Ex Tap (TAKARA) and a primer specific to each gene (IL-17, IL-6, TNF-α). Sequence of the primers used in the experiment are as follows (Table 14).

TABLE 14

| Target Gene | Base Sequence |
|---|---|
| mIL-17 | (F) 5′-TCCACCGCAATGAAGACCCTGATA-3′ |
| | (SEQ ID NO: 3) |
| | (R) 5′-ACCAGCATCTTCTCGACCCTGAAA-3′ |
| | (SEQ ID NO: 2) |
| mIL-6 | (F) 5′-TGGCTAAGGACCAAGACCAT-3′ |
| | (SEQ ID NO: 3) |
| | (R) 5′-TAACGCACTAGGTTTGCCGA-3′ |
| | (SEQ ID NO: 4) |
| TNF-α | (F) 5′-AGCCGATGGGTTGTACCTTGTCTA-3′ |
| | (SEQ ID NO: 5) |
| | (R) 5′-TGAGATAGCAAATCGGCTGACGGT-3′ |
| | (SEQ ID NO: 6) |

As a result, it was identified that mRNA expression levels of inflammatory cytokines, i.e., IL-17, IL-6 and TNF-α are all considerably increased in the mouse of induced dry eye dosed with the vehicle-1 through eye drops or not, but expression levels of IL-17, IL-6 and TNF-α are remarkably decreased in the mouse dosed with the compound of the present invention through eye drops to the same level of the normal mouse (FIG. 3). The experimental results above show that the administration of the compound of the present invention for dry eye effectively decreases the level of inflammatory cytokines, in particular, IL-6 known to have correlationship with a severity of dry eye, such that the composition of the present invention may be utilized as an effective therapeutic agent for dry eye.

While specific portions of the present invention have been described in detail above, it is apparent to those skilled in the art that such detailed descriptions are set forth to illustrate preferred exemplary embodiments only, but not construed to limit the scope of the present invention. Thus, it should be understood that the substantial scope of the present invention is defined by the accompanying claims and equivalents thereto.

1. A pharmaceutical composition for preventing or treating dry eye, comprising a compound represented by a following formula I, an optical isomer thereof or a pharmaceutically acceptable salt thereof as an effective component: 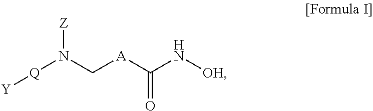
wherein
A is 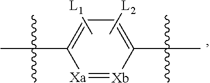
Xa and Xb are each independently CH or N,
L₁ and L₂ are each independently hydrogen, halogen, —CF₃ or —C₁₋₃ straight or branched chain alkyl,
Q is C(═O), S(═O)₂, S(═O) or C(═NH),
Y is selected from a following group: 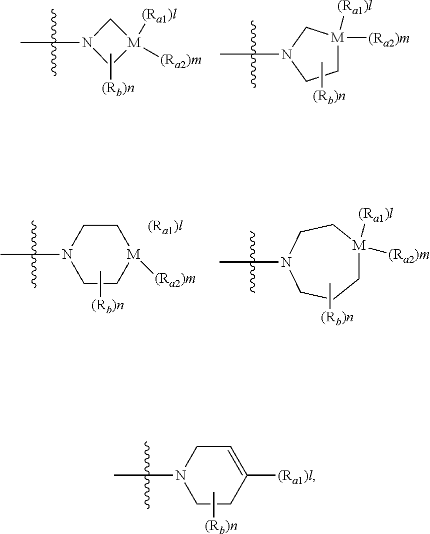
M is C, N, O, S or S(═O)₂, wherein, at this time, in case M is C, 1 and m are 1; in case M is N, 1 is 1 and m is 0; and in case M is O, S or S(═O)₂, 1 and m are 0,
Rₐₗ and Rₐ₂ are each independently hydrogen; hydroxy; —C₁₋₄ straight or branched chain alkyl, which is unsubstituted or substituted with at least one halogen; —C₁₋₄ straight or branched chain alcohol; benzhydryl;
—C₁₋₄ straight or branched chain alkyl, which is substituted with a saturated or unsaturated 5- to 7-membered heterocyclized compound comprising 1 to 3 heteroatoms of N, O or S as a ring member, wherein, at this time, the heterocyclized compound may be unsubstituted or at least one hydrogen may be optionally substituted with OH, OCH₃, CH₃, CH₂CH₃ or halogen; a saturated or unsaturated 5- to 7-membered heterocyclized compound comprising 1 to 3 heteroatoms of N, O or S as a ring member, wherein, at this time, the heterocyclized compound may be unsubstituted or at least one hydrogen may be optionally substituted with OH, OCH₃, CH₃, CH₂CH₃ or halogen; phenyl, wherein it is unsubstituted or at least one hydrogen is substituted with halogen, C₁₋₄ alkoxy, C₁₋₂ alkyl or hydroxy; benzyl, wherein it is unsubstituted or at least one hydrogen is substituted with halogen, C₁₋₄ alkoxy, C₁₋₂ alkyl or hydroxy; —S(═O)₂CH₃; halogen; —C₁₋₆ straight or branched chain alkoxy; —C₂₋₆ alkoxyalkyl; —C(═O)Rₓ, wherein Rₓ is straight or branched chain C₁₋₃ alkyl or C₃₋₁₀ cycloalkyl; 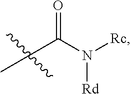
wherein R_{c} and R_{d} are each independently hydrogen, C₁₋₃ straight or branched chain alkyl; and 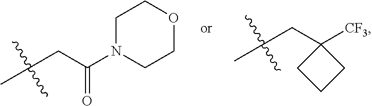 [SMILES: *CC(=O)N1CCOCC1 ; *CC1(C(F)(F)F)CCC1]
n is an integer of 0, 1 or 2,
R_{b} is hydrogen; hydroxy; —C₁₋₆ straight or branched chain alkyl, wherein it is unsubstituted or at least one hydrogen is substituted with halogen; —C(═O)CH₃; —C₁₋₄ straight or branched chain hydroxyalkyl; —C₁₋₆ straight or branched chain alkoxy; —C₂₋₆ straight or branched chain alkoxyalkyl; —CF₃; halogen; or 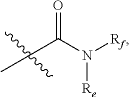
Rₑ and R_{f} are each independently hydrogen or —C₁₋₃ straight or branched chain alkyl,
Z is selected from a following group: 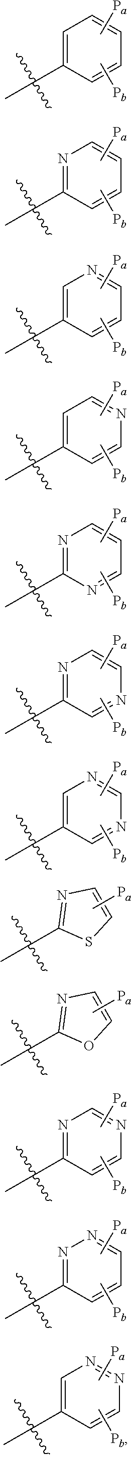
Pₐ and P_{b} are each independently 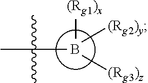
hydrogen; hydroxy; C₁₋₄ straight or branched chain alkyl, wherein it is unsubstituted or at least one hydrogen is substituted with halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol,
here 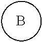
is phenyl, pyridine, pyrimidine, thiazole, indole, indazole, piperazine, quinoline, furan, tetrahydropyridine, piperidine or a ring selected from a following group: 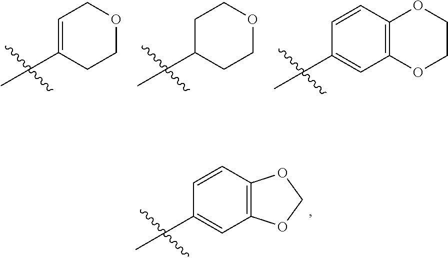 [SMILES: *C1=CCOCC1 ; *C1CCOCC1 ; *c1ccc2c(c1)OCCO2 ; *c1ccc2c(c1)OCO2]
x, y and z are each independently an integer of 0 or 1,
R_{g1}, R_{g2} and R_{g3} are each independently hydrogen; hydroxy; —C₁₋₃ alkyl; —CF₃; —C₁₋₆ straight or branched chain alkoxy; —C₂₋₆ straight or branched chain alkyl alkoxy; —C(=O)CH₃; —C₁₋₄ straight or branched chain hydroxyalkyl; —N(CH₃)₂; halogen; phenyl; —S((═O)₂)CH₃; or selected from a following group:

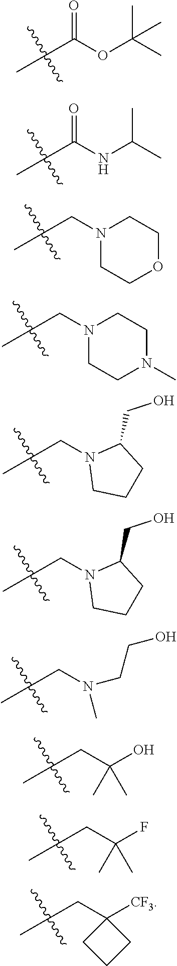

2. The pharmaceutical composition, according to claim 1, wherein the compound represented by the formula I above is a compound described in a following table:
TABLE 1

| Com- | |
|---|---|
| pound | Structure |
| 252 | 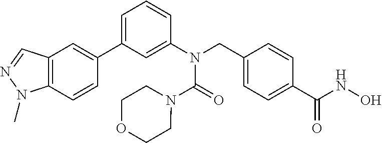 [SMILES: Cn1ncc2cc(-c3cccc(N(Cc4ccc(C(=O)NO)cc4)C(=O)N4CCOCC4)c3)ccc21] |
| 253 | 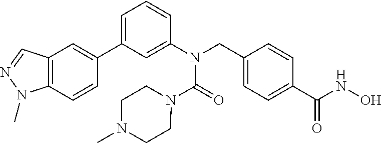 [SMILES: CN1CCN(C(=O)N(Cc2ccc(C(=O)NO)cc2)c2cccc(-c3ccc4c(cnn4C)c3)c2)CC1] |
| 254 | 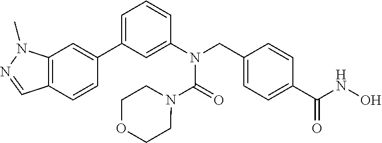 [SMILES: Cn1ncc2ccc(-c3cccc(N(Cc4ccc(C(=O)NO)cc4)C(=O)N4CCOCC4)c3)cc21] |
| 255 | 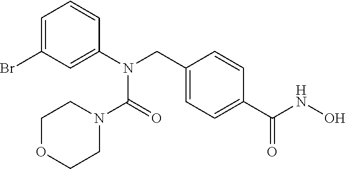 [SMILES: O=C(NO)c1ccc(CN(C(=O)N2CCOCC2)c2cccc(Br)c2)cc1] |
| 256 | 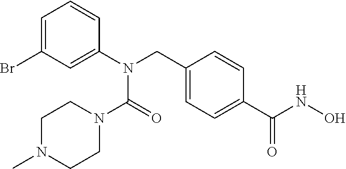 [SMILES: CN1CCN(C(=O)N(Cc2ccc(C(=O)NO)cc2)c2cccc(Br)c2)CC1] |
| 260 | 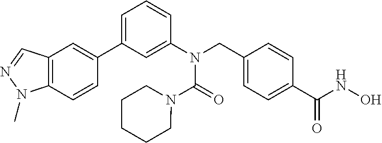 [SMILES: Cn1ncc2cc(-c3cccc(N(Cc4ccc(C(=O)NO)cc4)C(=O)N4CCCCC4)c3)ccc21] |
| 261 | 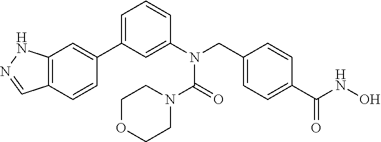 [SMILES: O=C(NO)c1ccc(CN(C(=O)N2CCOCC2)c2cccc(-c3ccc4cn[nH]c4c3)c2)cc1] |
| 262 | 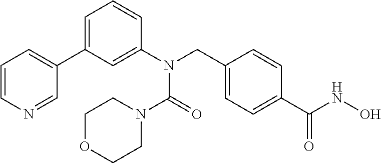 [SMILES: O=C(NO)c1ccc(CN(C(=O)N2CCOCC2)c2cccc(-c3cccnc3)c2)cc1] |
| 263 | 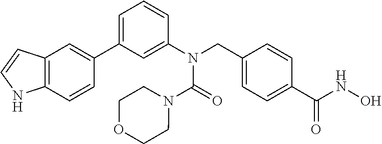 [SMILES: O=C(NO)c1ccc(CN(C(=O)N2CCOCC2)c2cccc(-c3ccc4[nH]ccc4c3)c2)cc1] |
| 279 | 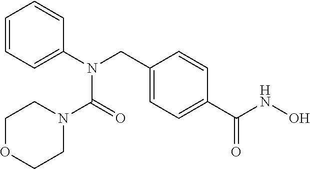 [SMILES: O=C(NO)c1ccc(CN(C(=O)N2CCOCC2)c2ccccc2)cc1] |
| 280 | 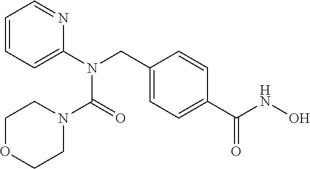 [SMILES: O=C(NO)c1ccc(CN(C(=O)N2CCOCC2)c2ccccn2)cc1] |
| 281 | 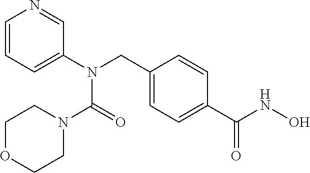 [SMILES: O=C(NO)c1ccc(CN(C(=O)N2CCOCC2)c2cccnc2)cc1] |
| 309 | 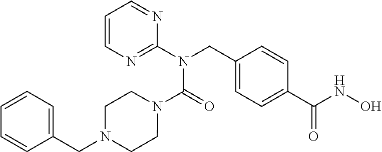 [SMILES: O=C(NO)c1ccc(CN(C(=O)N2CCN(Cc3ccccc3)CC2)c2ncccn2)cc1] |
| 311 | 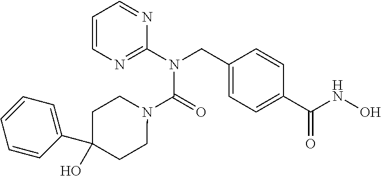 [SMILES: O=C(NO)c1ccc(CN(C(=O)N2CCC(O)(c3ccccc3)CC2)c2ncccn2)cc1] |

TABLE 2

| Compound | Structure |
|---|---|
| 312 | 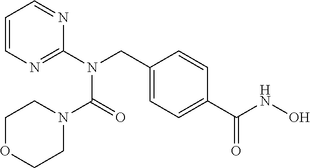 [SMILES: O=C(NO)c1ccc(CN(C(=O)N2CCOCC2)c2ncccn2)cc1] |
| 313 | 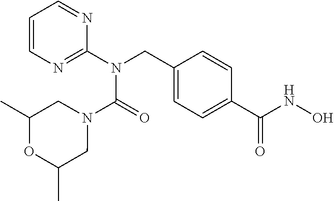 [SMILES: CC1CN(C(=O)N(Cc2ccc(C(=O)NO)cc2)c2ncccn2)CC(C)O1] |
| 329 | 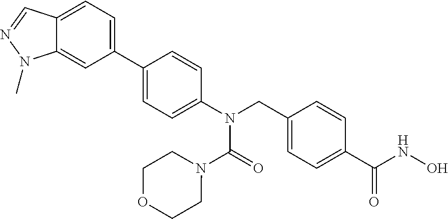 [SMILES: Cn1ncc2ccc(-c3ccc(N(Cc4ccc(C(=O)NO)cc4)C(=O)N4CCOCC4)cc3)cc21] |
| 330 | 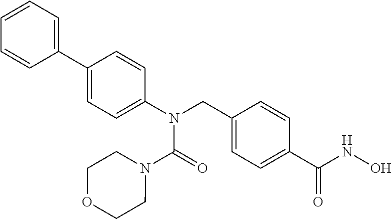 [SMILES: O=C(NO)c1ccc(CN(C(=O)N2CCOCC2)c2ccc(-c3ccccc3)cc2)cc1] |
| 331 | 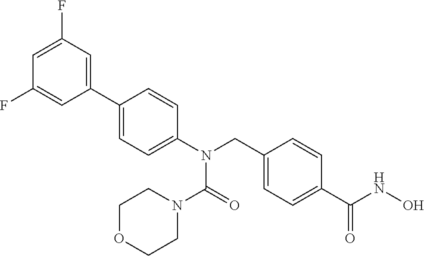 [SMILES: O=C(NO)c1ccc(CN(C(=O)N2CCOCC2)c2ccc(-c3cc(F)cc(F)c3)cc2)cc1] |
| 332 | 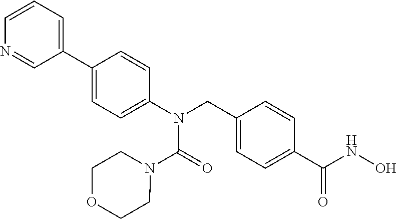 [SMILES: O=C(NO)c1ccc(CN(C(=O)N2CCOCC2)c2ccc(-c3cccnc3)cc2)cc1] |
| 333 | 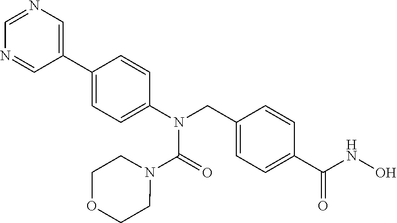 [SMILES: O=C(NO)c1ccc(CN(C(=O)N2CCOCC2)c2ccc(-c3cncnc3)cc2)cc1] |
| 334 | 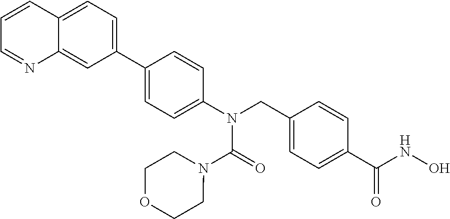 [SMILES: O=C(NO)c1ccc(CN(C(=O)N2CCOCC2)c2ccc(-c3ccc4cccnc4c3)cc2)cc1] |
| 335 | 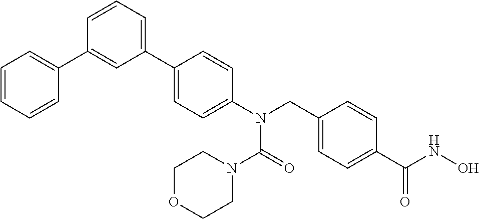 [SMILES: O=C(NO)c1ccc(CN(C(=O)N2CCOCC2)c2ccc(-c3cccc(-c4ccccc4)c3)cc2)cc1] |
| 336 | 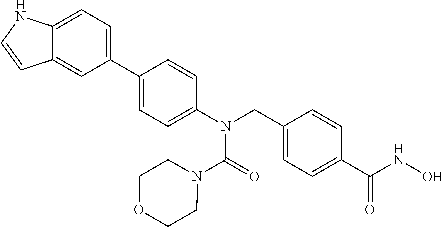 [SMILES: O=C(NO)c1ccc(CN(C(=O)N2CCOCC2)c2ccc(-c3ccc4[nH]ccc4c3)cc2)cc1] |
| 337 | 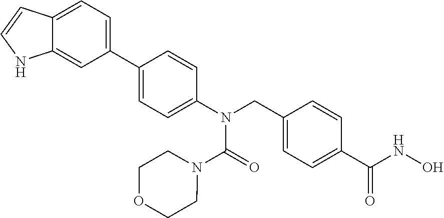 [SMILES: O=C(NO)c1ccc(CN(C(=O)N2CCOCC2)c2ccc(-c3ccc4cc[nH]c4c3)cc2)cc1] |
| 338 | 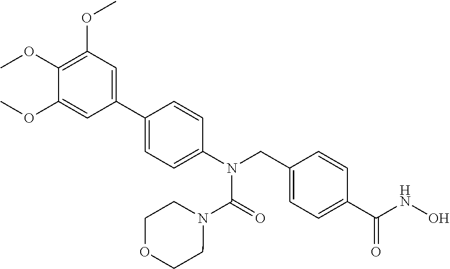 [SMILES: COc1cc(-c2ccc(N(Cc3ccc(C(=O)NO)cc3)C(=O)N3CCOCC3)cc2)cc(OC)c1OC] |
| 339 | 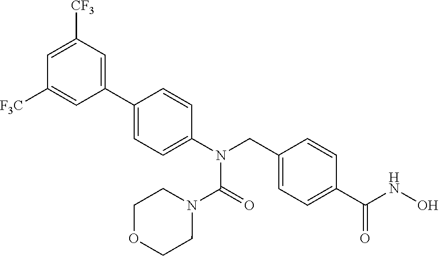 [SMILES: O=C(NO)c1ccc(CN(C(=O)N2CCOCC2)c2ccc(-c3cc(C(F)(F)F)cc(C(F)(F)F)c3)cc2)cc1] |
| 340 | 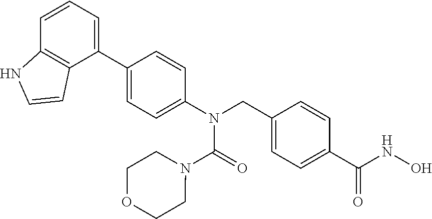 [SMILES: O=C(NO)c1ccc(CN(C(=O)N2CCOCC2)c2ccc(-c3cccc4[nH]ccc34)cc2)cc1] |

TABLE 3

| Com- | |
|---|---|
| pound | Structure |
| 341 | 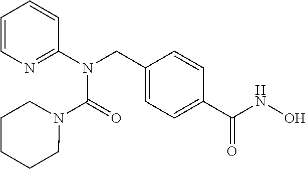 [SMILES: O=C(NO)c1ccc(CN(C(=O)N2CCCCC2)c2ccccn2)cc1] |
| 342 | 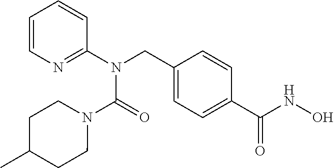 [SMILES: CC1CCN(C(=O)N(Cc2ccc(C(=O)NO)cc2)c2ccccn2)CC1] |
| 343 | 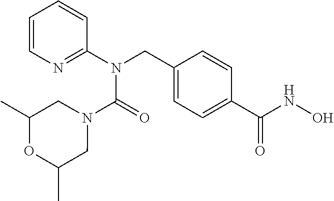 [SMILES: CC1CN(C(=O)N(Cc2ccc(C(=O)NO)cc2)c2ccccn2)CC(C)O1] |
| 352 | 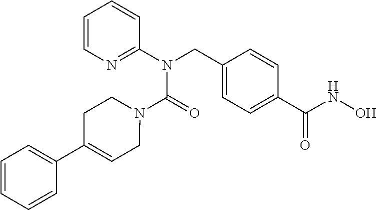 [SMILES: O=C(NO)c1ccc(CN(C(=O)N2CC=C(c3ccccc3)CC2)c2ccccn2)cc1] |
| 353 | 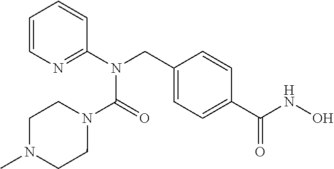 [SMILES: CN1CCN(C(=O)N(Cc2ccc(C(=O)NO)cc2)c2ccccn2)CC1] |
| 354 | 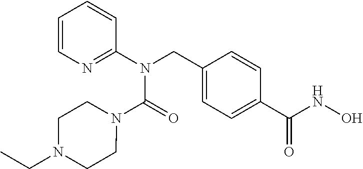 [SMILES: CCN1CCN(C(=O)N(Cc2ccc(C(=O)NO)cc2)c2ccccn2)CC1] |
| 355 | 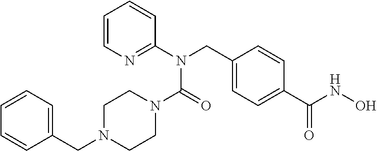 [SMILES: O=C(NO)c1ccc(CN(C(=O)N2CCN(Cc3ccccc3)CC2)c2ccccn2)cc1] |
| 356 | 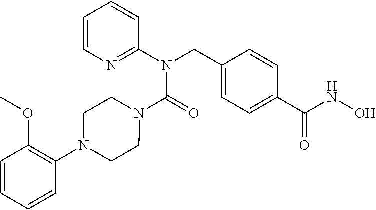 [SMILES: COc1ccccc1N1CCN(C(=O)N(Cc2ccc(C(=O)NO)cc2)c2ccccn2)CC1] |
| 357 | 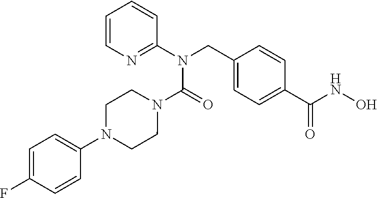 [SMILES: O=C(NO)c1ccc(CN(C(=O)N2CCN(c3ccc(F)cc3)CC2)c2ccccn2)cc1] |
| 358 | 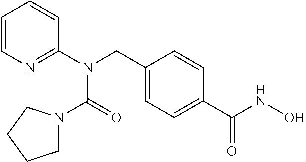 [SMILES: O=C(NO)c1ccc(CN(C(=O)N2CCCC2)c2ccccn2)cc1] |
| 370 | 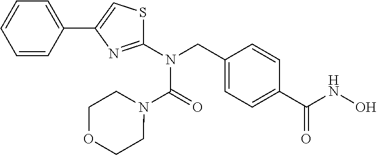 [SMILES: O=C(NO)c1ccc(CN(C(=O)N2CCOCC2)c2nc(-c3ccccc3)cs2)cc1] |
| 371 | 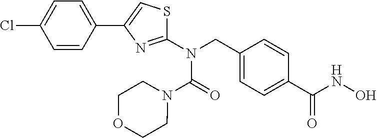 [SMILES: O=C(NO)c1ccc(CN(C(=O)N2CCOCC2)c2nc(-c3ccc(Cl)cc3)cs2)cc1] |
| 372 | 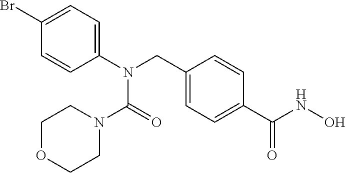 [SMILES: O=C(NO)c1ccc(CN(C(=O)N2CCOCC2)c2ccc(Br)cc2)cc1] |
| 374 | 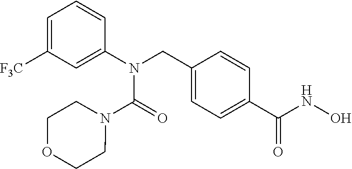 [SMILES: O=C(NO)c1ccc(CN(C(=O)N2CCOCC2)c2cccc(C(F)(F)F)c2)cc1] |

TABLE 4

| Compound | Structure |
|---|---|
| 376 | 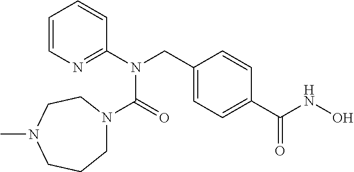 [SMILES: CN1CCCN(C(=O)N(Cc2ccc(C(=O)NO)cc2)c2ccccn2)CC1] |
| 377 | 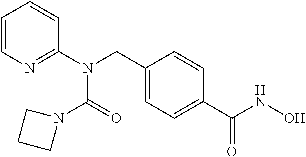 [SMILES: O=C(NO)c1ccc(CN(C(=O)N2CCC2)c2ccccn2)cc1] |
| 379 | 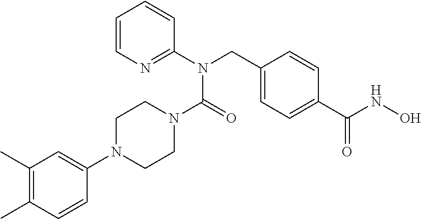 [SMILES: Cc1ccc(N2CCN(C(=O)N(Cc3ccc(C(=O)NO)cc3)c3ccccn3)CC2)cc1C] |
| 380 | 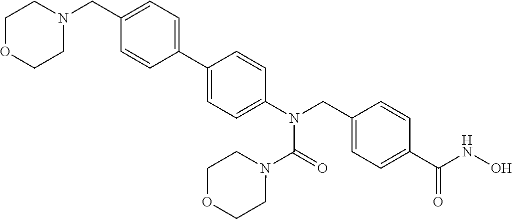 [SMILES: O=C(NO)c1ccc(CN(C(=O)N2CCOCC2)c2ccc(-c3ccc(CN4CCOCC4)cc3)cc2)cc1] |
| 381 | 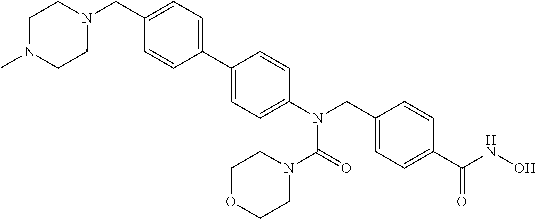 [SMILES: CN1CCN(Cc2ccc(-c3ccc(N(Cc4ccc(C(=O)NO)cc4)C(=O)N4CCOCC4)cc3)cc2)CC1] |
| 382 | 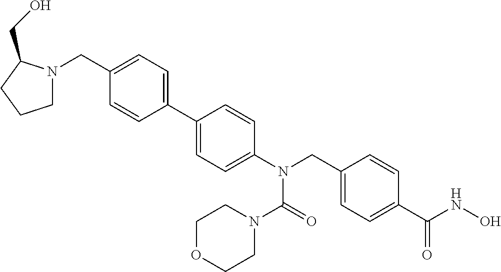 [SMILES: O=C(NO)c1ccc(CN(C(=O)N2CCOCC2)c2ccc(-c3ccc(CN4CCC[C@H]4CO)cc3)cc2)cc1] |
| 383 | 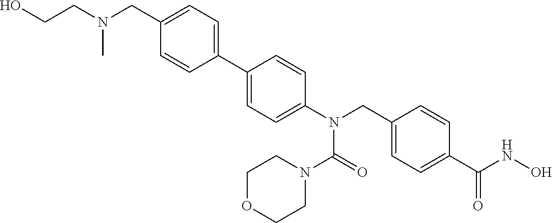 [SMILES: CN(CCO)Cc1ccc(-c2ccc(N(Cc3ccc(C(=O)NO)cc3)C(=O)N3CCOCC3)cc2)cc1] |
| 385 | 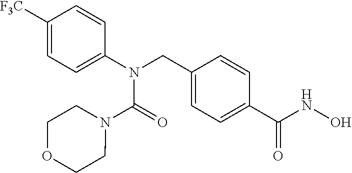 [SMILES: O=C(NO)c1ccc(CN(C(=O)N2CCOCC2)c2ccc(C(F)(F)F)cc2)cc1] |
| 386 | 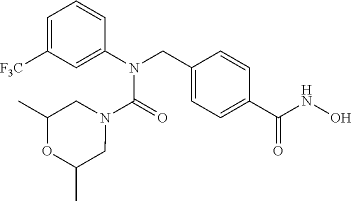 [SMILES: CC1CN(C(=O)N(Cc2ccc(C(=O)NO)cc2)c2cccc(C(F)(F)F)c2)CC(C)O1] |
| 389 | 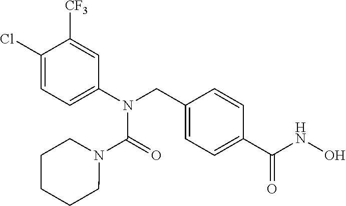 [SMILES: O=C(NO)c1ccc(CN(C(=O)N2CCCCC2)c2ccc(Cl)c(C(F)(F)F)c2)cc1] |
| 390 | 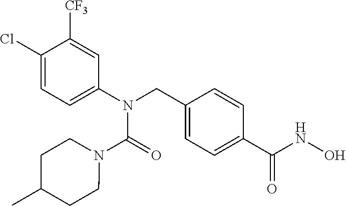 [SMILES: CC1CCN(C(=O)N(Cc2ccc(C(=O)NO)cc2)c2ccc(Cl)c(C(F)(F)F)c2)CC1] |
| 391 | 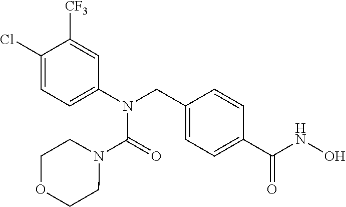 [SMILES: O=C(NO)c1ccc(CN(C(=O)N2CCOCC2)c2ccc(Cl)c(C(F)(F)F)c2)cc1] |
| 392 | 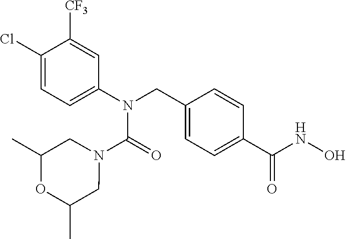 [SMILES: CC1CN(C(=O)N(Cc2ccc(C(=O)NO)cc2)c2ccc(Cl)c(C(F)(F)F)c2)CC(C)O1] |
| 393 | 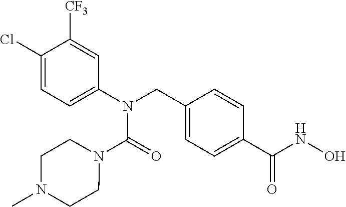 [SMILES: CN1CCN(C(=O)N(Cc2ccc(C(=O)NO)cc2)c2ccc(Cl)c(C(F)(F)F)c2)CC1] |

TABLE 5

| Compound | Structure |
|---|---|
| 394 | 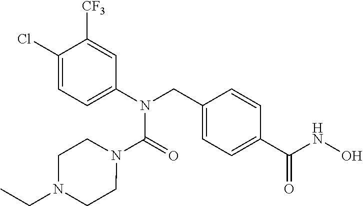 [SMILES: CCN1CCN(C(=O)N(Cc2ccc(C(=O)NO)cc2)c2ccc(Cl)c(C(F)(F)F)c2)CC1] |
| 395 | 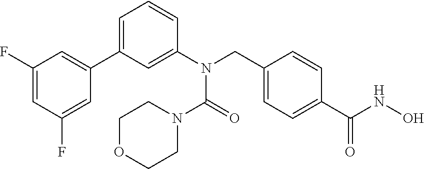 [SMILES: O=C(NO)c1ccc(CN(C(=O)N2CCOCC2)c2cccc(-c3cc(F)cc(F)c3)c2)cc1] |
| 396 | 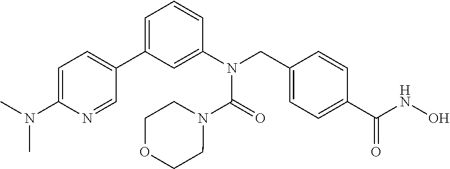 [SMILES: CN(C)c1ccc(-c2cccc(N(Cc3ccc(C(=O)NO)cc3)C(=O)N3CCOCC3)c2)cn1] |
| 397 | 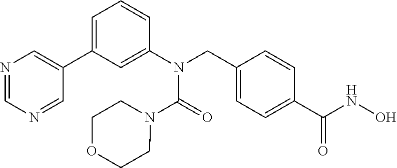 [SMILES: O=C(NO)c1ccc(CN(C(=O)N2CCOCC2)c2cccc(-c3cncnc3)c2)cc1] |
| 398 | 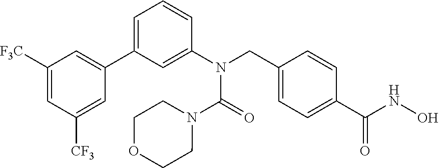 [SMILES: O=C(NO)c1ccc(CN(C(=O)N2CCOCC2)c2cccc(-c3cc(C(F)(F)F)cc(C(F)(F)F)c3)c2)cc1] |
| 399 | 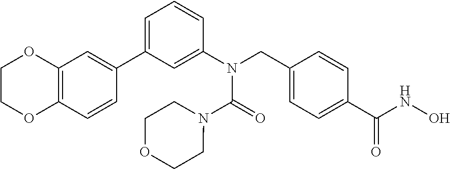 [SMILES: O=C(NO)c1ccc(CN(C(=O)N2CCOCC2)c2cccc(-c3ccc4c(c3)OCCO4)c2)cc1] |
| 400 | 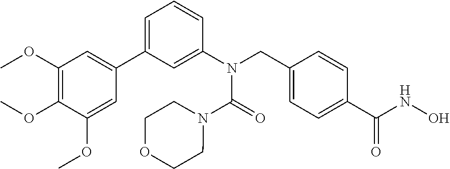 [SMILES: COc1cc(-c2cccc(N(Cc3ccc(C(=O)NO)cc3)C(=O)N3CCOCC3)c2)cc(OC)c1OC] |
| 401 | 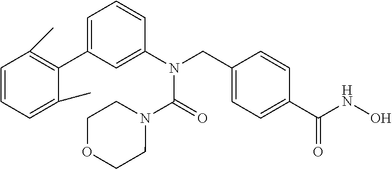 [SMILES: Cc1cccc(C)c1-c1cccc(N(Cc2ccc(C(=O)NO)cc2)C(=O)N2CCOCC2)c1] |
| 402 | 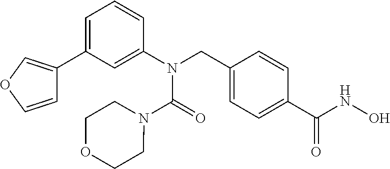 [SMILES: O=C(NO)c1ccc(CN(C(=O)N2CCOCC2)c2cccc(-c3ccoc3)c2)cc1] |
| 403 | 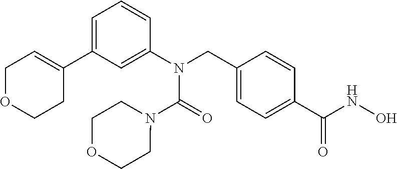 [SMILES: O=C(NO)c1ccc(CN(C(=O)N2CCOCC2)c2cccc(C3=CCOCC3)c2)cc1] |
| 404 | 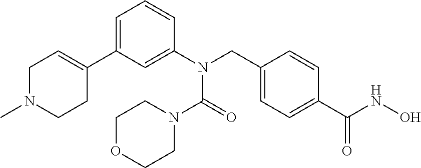 [SMILES: CN1CC=C(c2cccc(N(Cc3ccc(C(=O)NO)cc3)C(=O)N3CCOCC3)c2)CC1] |
| 405 | 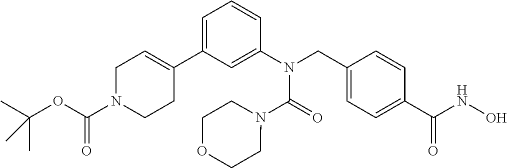 [SMILES: CC(C)(C)OC(=O)N1CC=C(c2cccc(N(Cc3ccc(C(=O)NO)cc3)C(=O)N3CCOCC3)c2)CC1] |
| 413 | 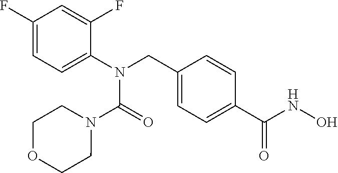 [SMILES: O=C(NO)c1ccc(CN(C(=O)N2CCOCC2)c2ccc(F)cc2F)cc1] |
| 414 | 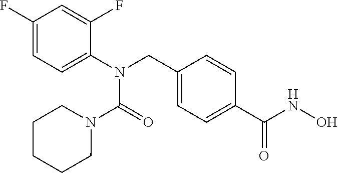 [SMILES: O=C(NO)c1ccc(CN(C(=O)N2CCCCC2)c2ccc(F)cc2F)cc1] |

TABLE 6

| Com- | |
|---|---|
| pound | Structure |
| 415 | 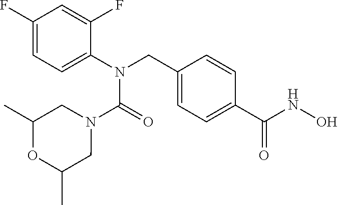 [SMILES: CC1CN(C(=O)N(Cc2ccc(C(=O)NO)cc2)c2ccc(F)cc2F)CC(C)O1] |
| 416 | 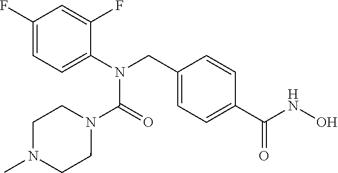 [SMILES: CN1CCN(C(=O)N(Cc2ccc(C(=O)NO)cc2)c2ccc(F)cc2F)CC1] |
| 418 | 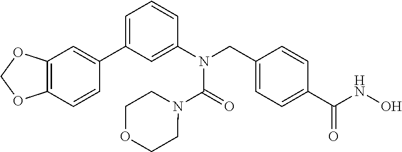 [SMILES: O=C(NO)c1ccc(CN(C(=O)N2CCOCC2)c2cccc(-c3ccc4c(c3)OCO4)c2)cc1] |
| 419 | 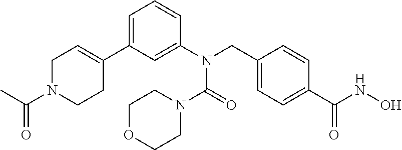 [SMILES: CC(=O)N1CC=C(c2cccc(N(Cc3ccc(C(=O)NO)cc3)C(=O)N3CCOCC3)c2)CC1] |
| 420 | 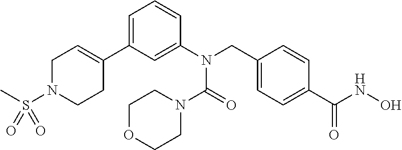 [SMILES: CS(=O)(=O)N1CC=C(c2cccc(N(Cc3ccc(C(=O)NO)cc3)C(=O)N3CCOCC3)c2)CC1] |
| 438 | 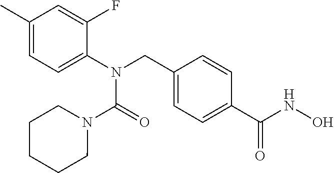 [SMILES: Cc1ccc(N(Cc2ccc(C(=O)NO)cc2)C(=O)N2CCCCC2)c(F)c1] |
| 439 | 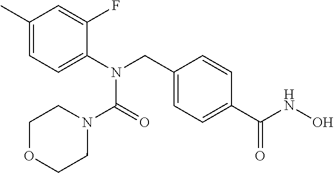 [SMILES: Cc1ccc(N(Cc2ccc(C(=O)NO)cc2)C(=O)N2CCOCC2)c(F)c1] |
| 440 | 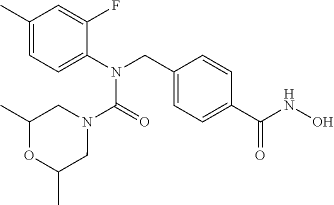 [SMILES: Cc1ccc(N(Cc2ccc(C(=O)NO)cc2)C(=O)N2CC(C)OC(C)C2)c(F)c1] |
| 441 | 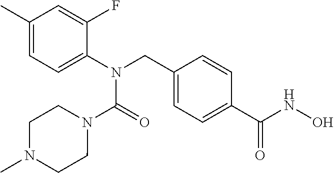 [SMILES: Cc1ccc(N(Cc2ccc(C(=O)NO)cc2)C(=O)N2CCN(C)CC2)c(F)c1] |
| 450 | 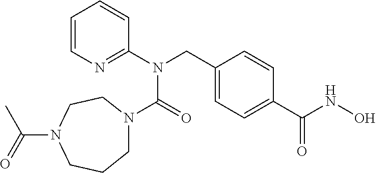 [SMILES: CC(=O)N1CCCN(C(=O)N(Cc2ccc(C(=O)NO)cc2)c2ccccn2)CC1] |
| 451 | 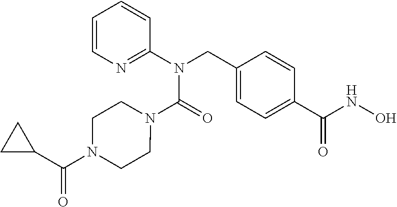 [SMILES: O=C(NO)c1ccc(CN(C(=O)N2CCN(C(=O)C3CC3)CC2)c2ccccn2)cc1] |
| 453 | 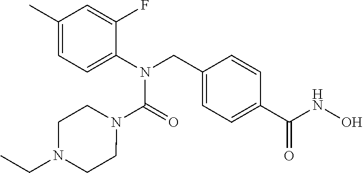 [SMILES: CCN1CCN(C(=O)N(Cc2ccc(C(=O)NO)cc2)c2ccc(C)cc2F)CC1] |
| 454 | 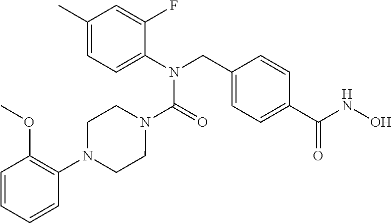 [SMILES: COc1ccccc1N1CCN(C(=O)N(Cc2ccc(C(=O)NO)cc2)c2ccc(C)cc2F)CC1] |
| 455 | 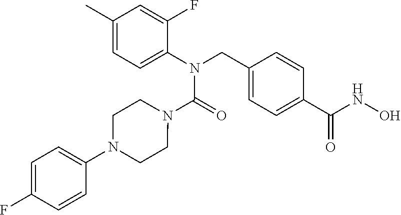 [SMILES: Cc1ccc(N(Cc2ccc(C(=O)NO)cc2)C(=O)N2CCN(c3ccc(F)cc3)CC2)c(F)c1] |

TABLE 7

| Com- | |
|---|---|
| pound | Structure |
| 456 | 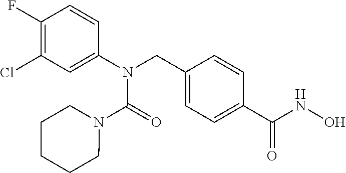 [SMILES: O=C(NO)c1ccc(CN(C(=O)N2CCCCC2)c2ccc(F)c(Cl)c2)cc1] |
| 457 | 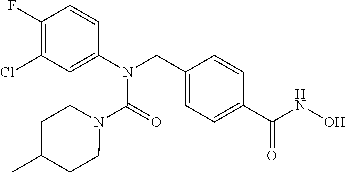 [SMILES: CC1CCN(C(=O)N(Cc2ccc(C(=O)NO)cc2)c2ccc(F)c(Cl)c2)CC1] |
| 458 | 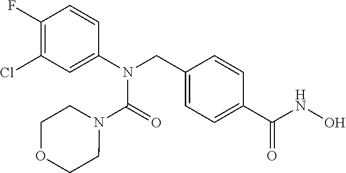 [SMILES: O=C(NO)c1ccc(CN(C(=O)N2CCOCC2)c2ccc(F)c(Cl)c2)cc1] |
| 459 | 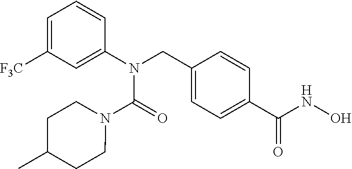 [SMILES: CC1CCN(C(=O)N(Cc2ccc(C(=O)NO)cc2)c2cccc(C(F)(F)F)c2)CC1] |
| 460 | 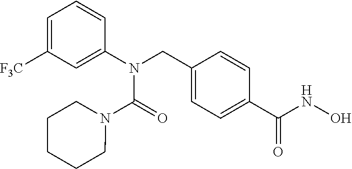 [SMILES: O=C(NO)c1ccc(CN(C(=O)N2CCCCC2)c2cccc(C(F)(F)F)c2)cc1] |
| 461 | 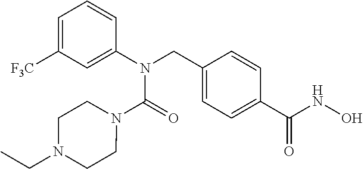 [SMILES: CCN1CCN(C(=O)N(Cc2ccc(C(=O)NO)cc2)c2cccc(C(F)(F)F)c2)CC1] |
| 462 | 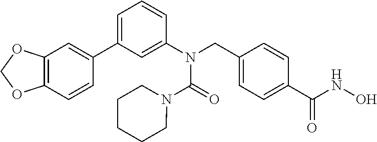 [SMILES: O=C(NO)c1ccc(CN(C(=O)N2CCCCC2)c2cccc(-c3ccc4c(c3)OCO4)c2)cc1] |
| 463 | 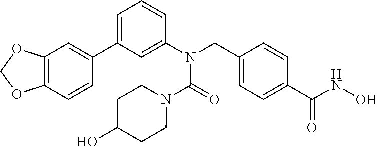 [SMILES: O=C(NO)c1ccc(CN(C(=O)N2CCC(O)CC2)c2cccc(-c3ccc4c(c3)OCO4)c2)cc1] |
| 464 | 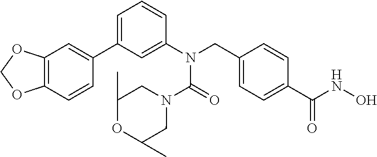 [SMILES: CC1CN(C(=O)N(Cc2ccc(C(=O)NO)cc2)c2cccc(-c3ccc4c(c3)OCO4)c2)CC(C)O1] |
| 465 | 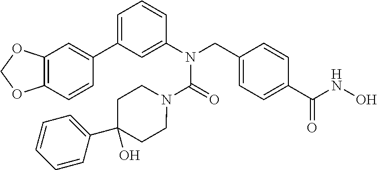 [SMILES: O=C(NO)c1ccc(CN(C(=O)N2CCC(O)(c3ccccc3)CC2)c2cccc(-c3ccc4c(c3)OCO4)c2)cc1] |
| 466 | 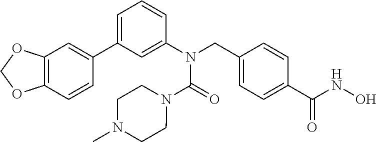 [SMILES: CN1CCN(C(=O)N(Cc2ccc(C(=O)NO)cc2)c2cccc(-c3ccc4c(c3)OCO4)c2)CC1] |
| 467 | 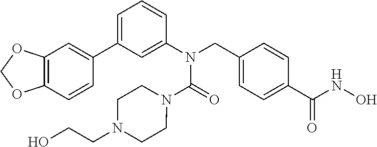 [SMILES: O=C(NO)c1ccc(CN(C(=O)N2CCN(CCO)CC2)c2cccc(-c3ccc4c(c3)OCO4)c2)cc1] |
| 468 | 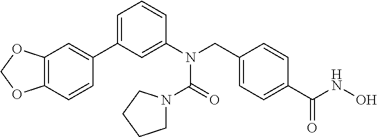 [SMILES: O=C(NO)c1ccc(CN(C(=O)N2CCCC2)c2cccc(-c3ccc4c(c3)OCO4)c2)cc1] |
| 469 | 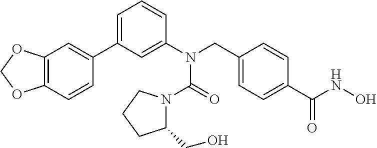 [SMILES: O=C(NO)c1ccc(CN(C(=O)N2CCC[C@H]2CO)c2cccc(-c3ccc4c(c3)OCO4)c2)cc1] |

TABLE 8

| Com- | |
|---|---|
| pound | Structure |
| 470 | 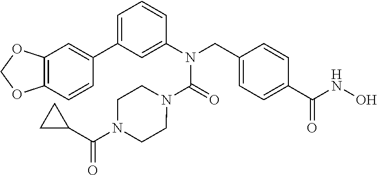 [SMILES: O=C(NO)c1ccc(CN(C(=O)N2CCN(C(=O)C3CC3)CC2)c2cccc(-c3ccc4c(c3)OCO4)c2)cc1] |
| 471 | 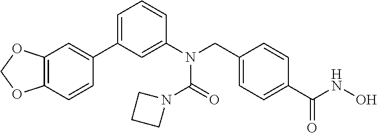 [SMILES: O=C(NO)c1ccc(CN(C(=O)N2CCC2)c2cccc(-c3ccc4c(c3)OCO4)c2)cc1] |
| 477 | 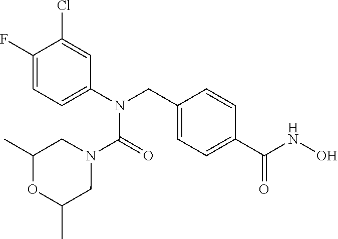 [SMILES: CC1CN(C(=O)N(Cc2ccc(C(=O)NO)cc2)c2ccc(F)c(Cl)c2)CC(C)O1] |
| 478 | 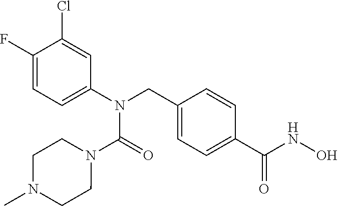 [SMILES: CN1CCN(C(=O)N(Cc2ccc(C(=O)NO)cc2)c2ccc(F)c(Cl)c2)CC1] |
| 479 | 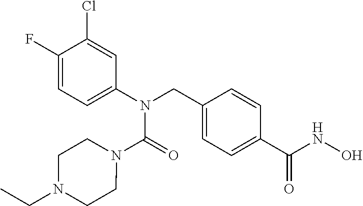 [SMILES: CCN1CCN(C(=O)N(Cc2ccc(C(=O)NO)cc2)c2ccc(F)c(Cl)c2)CC1] |
| 480 | 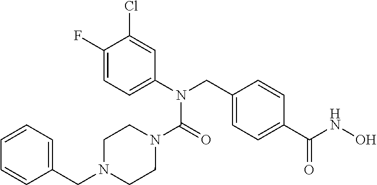 [SMILES: O=C(NO)c1ccc(CN(C(=O)N2CCN(Cc3ccccc3)CC2)c2ccc(F)c(Cl)c2)cc1] |
| 481 | 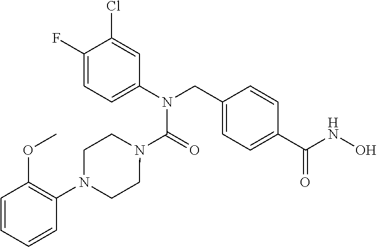 [SMILES: COc1ccccc1N1CCN(C(=O)N(Cc2ccc(C(=O)NO)cc2)c2ccc(F)c(Cl)c2)CC1] |
| 482 | 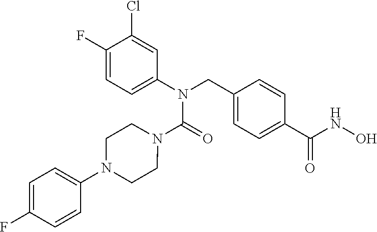 [SMILES: O=C(NO)c1ccc(CN(C(=O)N2CCN(c3ccc(F)cc3)CC2)c2ccc(F)c(Cl)c2)cc1] |
| 483 | 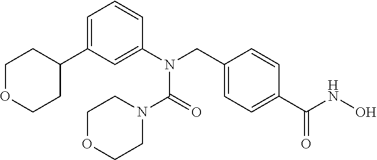 [SMILES: O=C(NO)c1ccc(CN(C(=O)N2CCOCC2)c2cccc(C3CCOCC3)c2)cc1] |
| 484 | 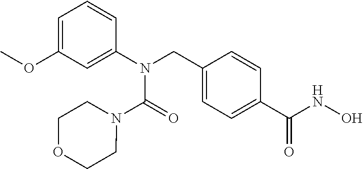 [SMILES: COc1cccc(N(Cc2ccc(C(=O)NO)cc2)C(=O)N2CCOCC2)c1] |
| 485 | 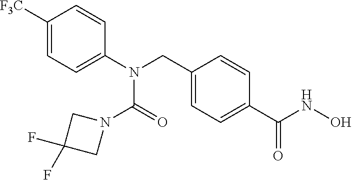 [SMILES: O=C(NO)c1ccc(CN(C(=O)N2CC(F)(F)C2)c2ccc(C(F)(F)F)cc2)cc1] |
| 486 | 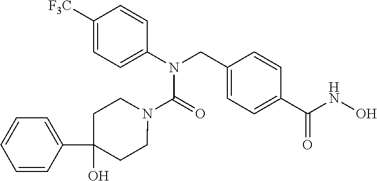 [SMILES: O=C(NO)c1ccc(CN(C(=O)N2CCC(O)(c3ccccc3)CC2)c2ccc(C(F)(F)F)cc2)cc1] |
| 487 | 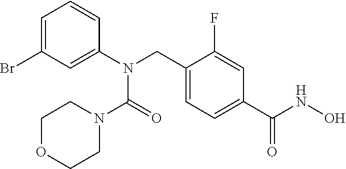 [SMILES: O=C(NO)c1ccc(CN(C(=O)N2CCOCC2)c2cccc(Br)c2)c(F)c1] |
| 488 | 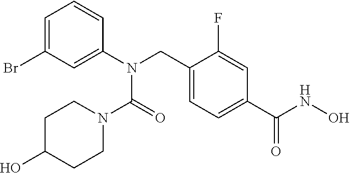 [SMILES: O=C(NO)c1ccc(CN(C(=O)N2CCC(O)CC2)c2cccc(Br)c2)c(F)c1] |

TABLE 9

| Compound | Structure |
|---|---|
| 489 | 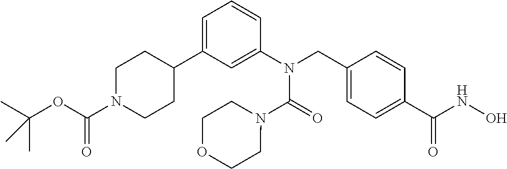 [SMILES: CC(C)(C)OC(=O)N1CCC(c2cccc(N(Cc3ccc(C(=O)NO)cc3)C(=O)N3CCOCC3)c2)CC1] |
| 490 | 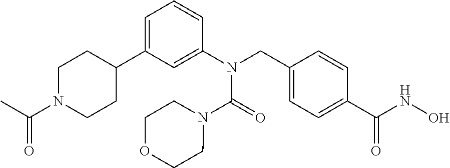 [SMILES: CC(=O)N1CCC(c2cccc(N(Cc3ccc(C(=O)NO)cc3)C(=O)N3CCOCC3)c2)CC1] |
| 491 | 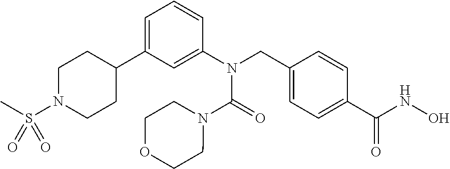 [SMILES: CS(=O)(=O)N1CCC(c2cccc(N(Cc3ccc(C(=O)NO)cc3)C(=O)N3CCOCC3)c2)CC1] |
| 492 | 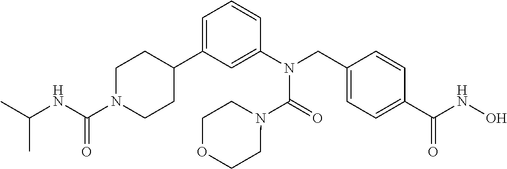 [SMILES: CC(C)NC(=O)N1CCC(c2cccc(N(Cc3ccc(C(=O)NO)cc3)C(=O)N3CCOCC3)c2)CC1] |
| 493 | 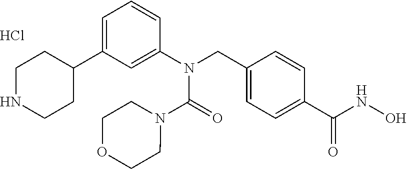 [SMILES: Cl ; O=C(NO)c1ccc(CN(C(=O)N2CCOCC2)c2cccc(C3CCNCC3)c2)cc1] |
| 494 | 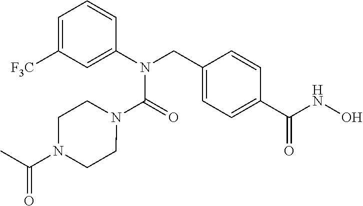 [SMILES: CC(=O)N1CCN(C(=O)N(Cc2ccc(C(=O)NO)cc2)c2cccc(C(F)(F)F)c2)CC1] |
| 495 | 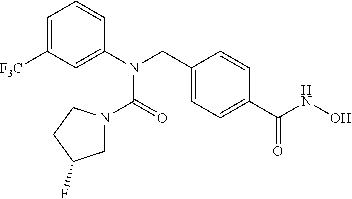 [SMILES: O=C(NO)c1ccc(CN(C(=O)N2CC[C@@H](F)C2)c2cccc(C(F)(F)F)c2)cc1] |
| 496 | 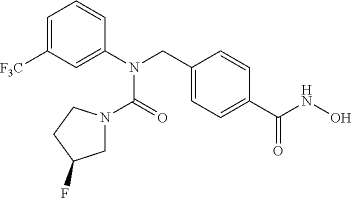 [SMILES: O=C(NO)c1ccc(CN(C(=O)N2CC[C@H](F)C2)c2cccc(C(F)(F)F)c2)cc1] |
| 497 | 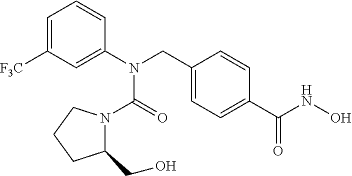 [SMILES: O=C(NO)c1ccc(CN(C(=O)N2CCC[C@@H]2CO)c2cccc(C(F)(F)F)c2)cc1] |
| 498 | 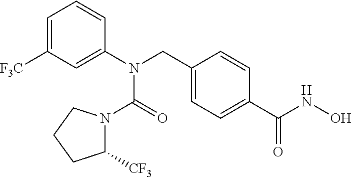 [SMILES: O=C(NO)c1ccc(CN(C(=O)N2CCC[C@H]2C(F)(F)F)c2cccc(C(F)(F)F)c2)cc1] |
| 499 | 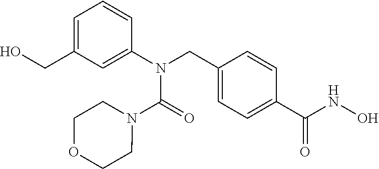 [SMILES: O=C(NO)c1ccc(CN(C(=O)N2CCOCC2)c2cccc(CO)c2)cc1] |
| 500 | 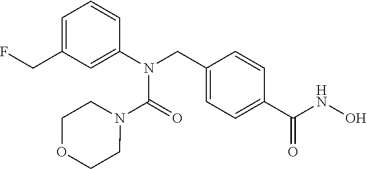 [SMILES: O=C(NO)c1ccc(CN(C(=O)N2CCOCC2)c2cccc(CF)c2)cc1] |
| 511 | 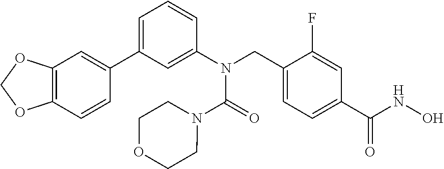 [SMILES: O=C(NO)c1ccc(CN(C(=O)N2CCOCC2)c2cccc(-c3ccc4c(c3)OCO4)c2)c(F)c1] |
| 512 | 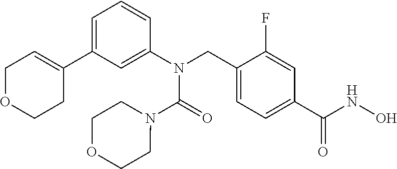 [SMILES: O=C(NO)c1ccc(CN(C(=O)N2CCOCC2)c2cccc(C3=CCOCC3)c2)c(F)c1] |

TABLE 10

| Compound | Structure |
|---|---|
| 513 | 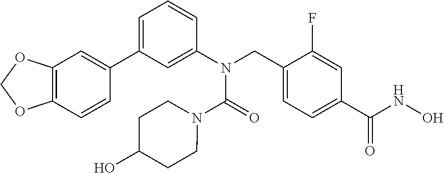 [SMILES: O=C(NO)c1ccc(CN(C(=O)N2CCC(O)CC2)c2cccc(-c3ccc4c(c3)OCO4)c2)c(F)c1] |
| 514 | 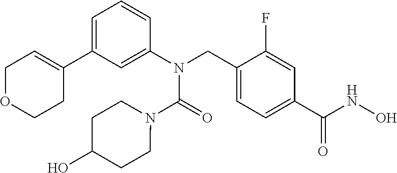 [SMILES: O=C(NO)c1ccc(CN(C(=O)N2CCC(O)CC2)c2cccc(C3=CCOCC3)c2)c(F)c1] |
| 517 | 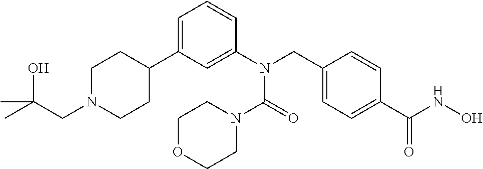 [SMILES: CC(C)(O)CN1CCC(c2cccc(N(Cc3ccc(C(=O)NO)cc3)C(=O)N3CCOCC3)c2)CC1] |
| 518 | 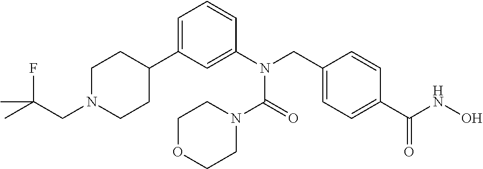 [SMILES: CC(C)(F)CN1CCC(c2cccc(N(Cc3ccc(C(=O)NO)cc3)C(=O)N3CCOCC3)c2)CC1] |
| 520 | 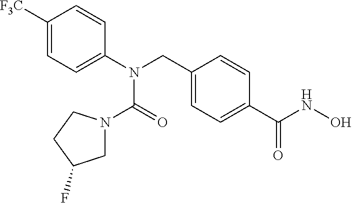 [SMILES: O=C(NO)c1ccc(CN(C(=O)N2CC[C@@H](F)C2)c2ccc(C(F)(F)F)cc2)cc1] |
| 521 | 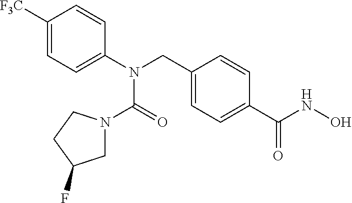 [SMILES: O=C(NO)c1ccc(CN(C(=O)N2CC[C@H](F)C2)c2ccc(C(F)(F)F)cc2)cc1] |
| 522 | 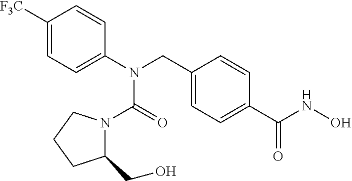 [SMILES: O=C(NO)c1ccc(CN(C(=O)N2CCC[C@@H]2CO)c2ccc(C(F)(F)F)cc2)cc1] |
| 529 | 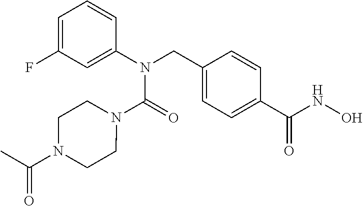 [SMILES: CC(=O)N1CCN(C(=O)N(Cc2ccc(C(=O)NO)cc2)c2cccc(F)c2)CC1] |
| 530 | 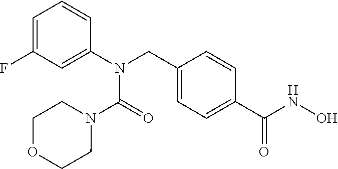 [SMILES: O=C(NO)c1ccc(CN(C(=O)N2CCOCC2)c2cccc(F)c2)cc1] |
| 531 | 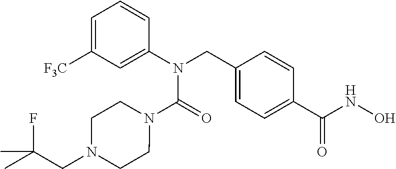 [SMILES: CC(C)(F)CN1CCN(C(=O)N(Cc2ccc(C(=O)NO)cc2)c2cccc(C(F)(F)F)c2)CC1] |
| 532 | 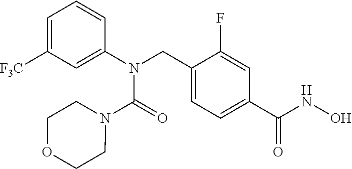 [SMILES: O=C(NO)c1ccc(CN(C(=O)N2CCOCC2)c2cccc(C(F)(F)F)c2)c(F)c1] |
| 533 | 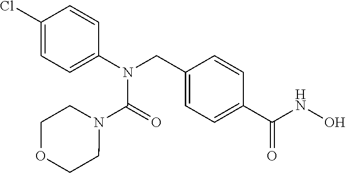 [SMILES: O=C(NO)c1ccc(CN(C(=O)N2CCOCC2)c2ccc(Cl)cc2)cc1] |
| 543 | 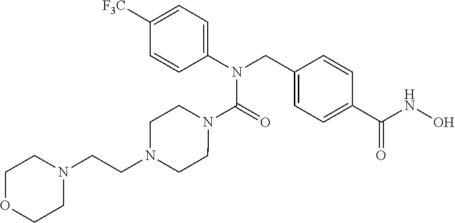 [SMILES: O=C(NO)c1ccc(CN(C(=O)N2CCN(CCN3CCOCC3)CC2)c2ccc(C(F)(F)F)cc2)cc1] |
| 544 | 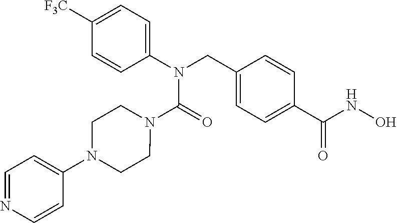 [SMILES: O=C(NO)c1ccc(CN(C(=O)N2CCN(c3ccncc3)CC2)c2ccc(C(F)(F)F)cc2)cc1] |

TABLE 11

| Com- | |
|---|---|
| pound | Structure |
| 545 | 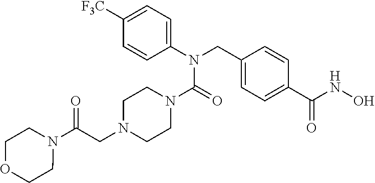 [SMILES: O=C(NO)c1ccc(CN(C(=O)N2CCN(CC(=O)N3CCOCC3)CC2)c2ccc(C(F)(F)F)cc2)cc1] |
| 577 | 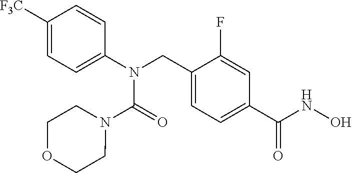 [SMILES: O=C(NO)c1ccc(CN(C(=O)N2CCOCC2)c2ccc(C(F)(F)F)cc2)c(F)c1] |
| 578 | 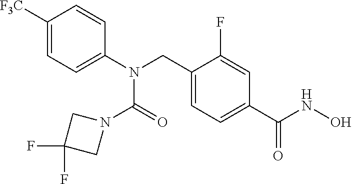 [SMILES: O=C(NO)c1ccc(CN(C(=O)N2CC(F)(F)C2)c2ccc(C(F)(F)F)cc2)c(F)c1] |
| 580 | 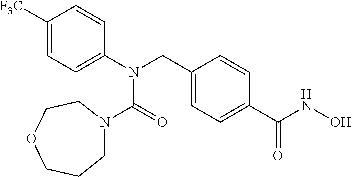 [SMILES: O=C(NO)c1ccc(CN(C(=O)N2CCCOCC2)c2ccc(C(F)(F)F)cc2)cc1] |
| 651 | 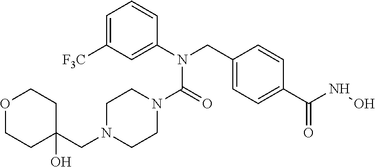 [SMILES: O=C(NO)c1ccc(CN(C(=O)N2CCN(CC3(O)CCOCC3)CC2)c2cccc(C(F)(F)F)c2)cc1] |
| 683 | 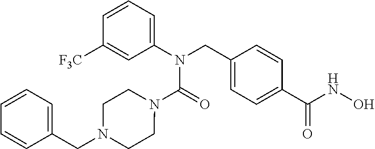 [SMILES: O=C(NO)c1ccc(CN(C(=O)N2CCN(Cc3ccccc3)CC2)c2cccc(C(F)(F)F)c2)cc1] |
| 684 | 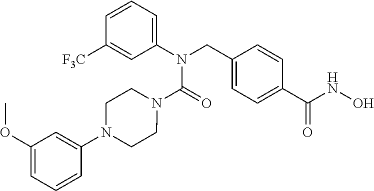 [SMILES: COc1cccc(N2CCN(C(=O)N(Cc3ccc(C(=O)NO)cc3)c3cccc(C(F)(F)F)c3)CC2)c1] |
| 716 | 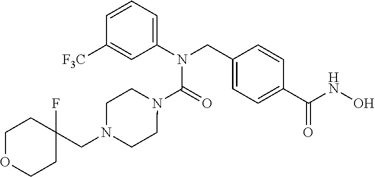 [SMILES: O=C(NO)c1ccc(CN(C(=O)N2CCN(CC3(F)CCOCC3)CC2)c2cccc(C(F)(F)F)c2)cc1] |
| 717 | 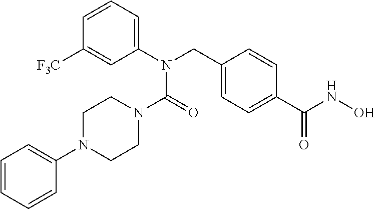 [SMILES: O=C(NO)c1ccc(CN(C(=O)N2CCN(c3ccccc3)CC2)c2cccc(C(F)(F)F)c2)cc1] |
| 718 | 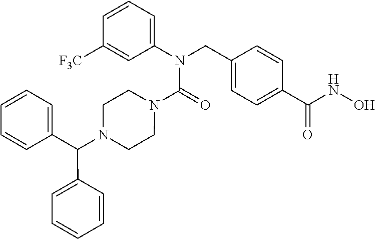 [SMILES: O=C(NO)c1ccc(CN(C(=O)N2CCN(C(c3ccccc3)c3ccccc3)CC2)c2cccc(C(F)(F)F)c2)cc1] |
| 765 | 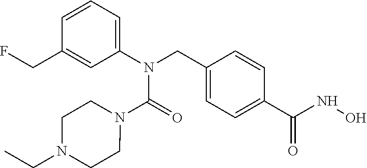 [SMILES: CCN1CCN(C(=O)N(Cc2ccc(C(=O)NO)cc2)c2cccc(CF)c2)CC1] |
| 766 | 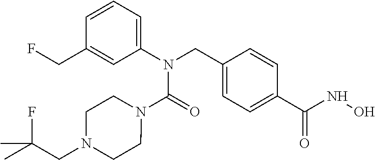 [SMILES: CC(C)(F)CN1CCN(C(=O)N(Cc2ccc(C(=O)NO)cc2)c2cccc(CF)c2)CC1] |
| 771 | 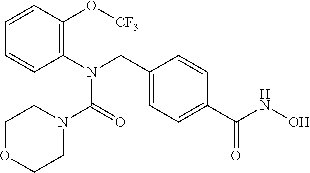 [SMILES: O=C(NO)c1ccc(CN(C(=O)N2CCOCC2)c2ccccc2OC(F)(F)F)cc1] |
| 772 | 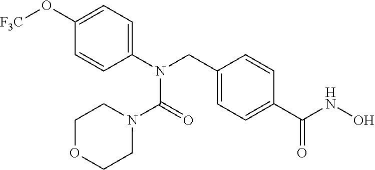 [SMILES: O=C(NO)c1ccc(CN(C(=O)N2CCOCC2)c2ccc(OC(F)(F)F)cc2)cc1] |

TABLE 12

| Com- | |
|---|---|
| pound | Structure |
| 773 | 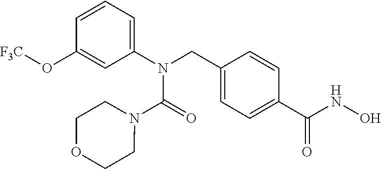 [SMILES: O=C(NO)c1ccc(CN(C(=O)N2CCOCC2)c2cccc(OC(F)(F)F)c2)cc1] |
| 774 | 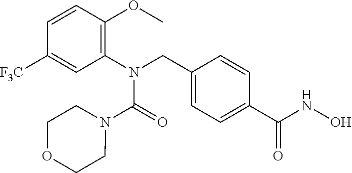 [SMILES: COc1ccc(C(F)(F)F)cc1N(Cc1ccc(C(=O)NO)cc1)C(=O)N1CCOCC1] |
| 776 | 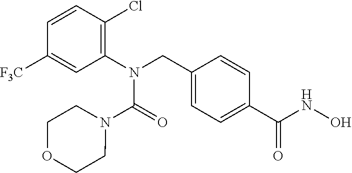 [SMILES: O=C(NO)c1ccc(CN(C(=O)N2CCOCC2)c2cc(C(F)(F)F)ccc2Cl)cc1] |
| 778 | 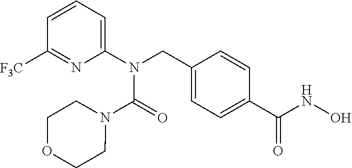 [SMILES: O=C(NO)c1ccc(CN(C(=O)N2CCOCC2)c2cccc(C(F)(F)F)n2)cc1] |
| 791 | 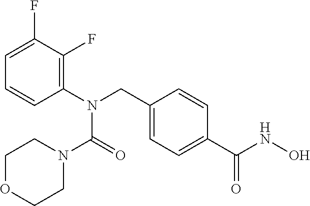 [SMILES: O=C(NO)c1ccc(CN(C(=O)N2CCOCC2)c2cccc(F)c2F)cc1] |
| 797 | 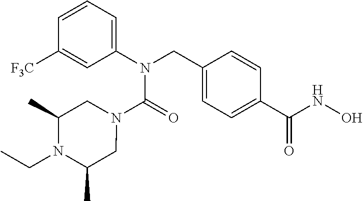 [SMILES: CCN1[C@H](C)CN(C(=O)N(Cc2ccc(C(=O)NO)cc2)c2cccc(C(F)(F)F)c2)C[C@@H]1C] |
| 800 | 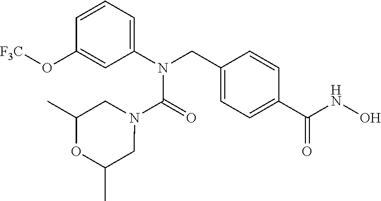 [SMILES: CC1CN(C(=O)N(Cc2ccc(C(=O)NO)cc2)c2cccc(OC(F)(F)F)c2)CC(C)O1] |
| 801 | 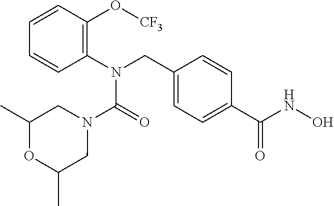 [SMILES: CC1CN(C(=O)N(Cc2ccc(C(=O)NO)cc2)c2ccccc2OC(F)(F)F)CC(C)O1] |
| 802 | 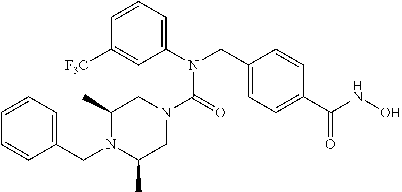 [SMILES: C[C@@H]1CN(C(=O)N(Cc2ccc(C(=O)NO)cc2)c2cccc(C(F)(F)F)c2)C[C@H](C)N1Cc1ccccc1] |
| 803 | 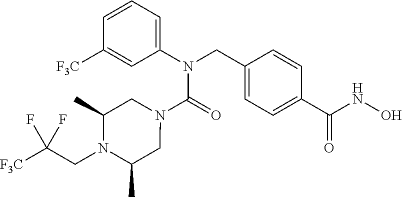 [SMILES: C[C@@H]1CN(C(=O)N(Cc2ccc(C(=O)NO)cc2)c2cccc(C(F)(F)F)c2)C[C@H](C)N1CC(F)(F)C(F)(F)F] |
| 826 | 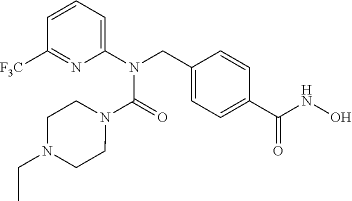 [SMILES: CCN1CCN(C(=O)N(Cc2ccc(C(=O)NO)cc2)c2cccc(C(F)(F)F)n2)CC1] |
| 827 | 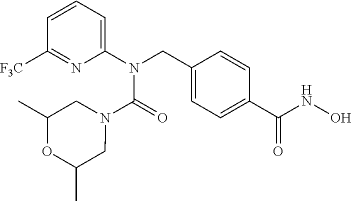 [SMILES: CC1CN(C(=O)N(Cc2ccc(C(=O)NO)cc2)c2cccc(C(F)(F)F)n2)CC(C)O1] |
| 828 | 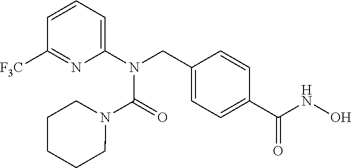 [SMILES: O=C(NO)c1ccc(CN(C(=O)N2CCCCC2)c2cccc(C(F)(F)F)n2)cc1] |
| 829 | 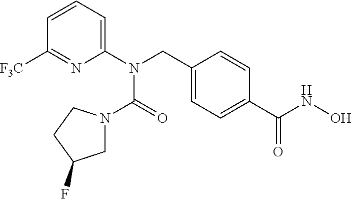 [SMILES: O=C(NO)c1ccc(CN(C(=O)N2CC[C@H](F)C2)c2cccc(C(F)(F)F)n2)cc1] |

3. The pharmaceutical composition, according to claim 1, wherein the said dry eye includes dry eye caused by Sjogren's syndrome or dry eye caused by non-Sjogren's syndrome.

4. The pharmaceutical composition, according to claim 1, wherein an expression of inflammatory cytokines is inhibited.

5. The pharmaceutical composition, according to claim 4, wherein the said inflammatory cytokines are IL-17, IL-6 or TNF-α.

6. The pharmaceutical composition, according to claim 1, wherein the pharmaceutical composition is locally administered.

7. The pharmaceutical composition, according to claim 6, wherein the said pharmaceutical composition is administered by eye drop.

8. The pharmaceutical composition, according to claim 1, wherein the said pharmaceutical composition is administered orally.

9. A method for treating dry eye, comprising administering a therapeutically effective amount of the compound represented by formula I, an optical isomer thereof or a pharmaceutically acceptable salt thereof according to claim 1.

10. Use of the compound represented by formula I, an optical isomer thereof or a pharmaceutically acceptable salt thereof according to claim 1, in the manufacture of a medicament for treating dry eye.


SEQUENCE LISTING (6 sequences)

SEQ ID NO: 1 - DNA, 24 bp, Artificial Sequence
  Pimer(F)_mIL-17
tccaccgcaatgaagaccctgata

SEQ ID NO: 2 - DNA, 24 bp, Artificial Sequence
  Pimer(R)_mIL-17
accagcatcttctcgaccctgaaa

SEQ ID NO: 3 - DNA, 20 bp, Artificial Sequence
  Pimer(F)_mIL-6
tggctaaggaccaagaccat

SEQ ID NO: 4 - DNA, 20 bp, Artificial Sequence
  Pimer(R)_mIL-6
taacgcactaggtttgccga

SEQ ID NO: 5 - DNA, 24 bp, Artificial Sequence
  Pimer(F)_TNF-alpha
agccgatgggttgtaccttgtcta

SEQ ID NO: 6 - DNA, 24 bp, Artificial Sequence
  Pimer(R)_TNF-alpha
tgagatagcaaatcggctgacggt